(19) United States
(12) Patent Application Publication
(10) Pub. No.: US 2025/0304680 A1
(43) Pub. Date: Oct. 2, 2025

(54) METHODS AND COMPOSITIONS FOR TREATING OR PREVENTING INFLAMMATORY SKIN DISORDERS

(71) Applicant: THE UAB RESEARCH FOUNDATION, Birmingham, AL (US)

(72) Inventors: Chander Raman, Birmingham, AL (US); Mohammad Athar, Birmingham, AL (US); Bharat Kumar Mishra, Birmingham, AL (US); Mahendra P. Kashyap, Birmingham, AL (US); Shahid M. Mukhtar, Birmingham, AL (US)

(73) Assignee: THE UAB RESEARCH FOUNDATION, Birmingham, AL (US)

(21) Appl. No.: 18/865,259

(22) PCT Filed: May 16, 2023
(86) PCT No.: PCT/US2023/022364

§ 371 Date: Nov. 12, 2024

Related U.S. Application Data
(60) Provisional application No. 63/342,886, filed on May 17, 2022.

(51) Int. Cl. C07K 16/28; A61P 17/10

(52) U.S. Cl. CPC C07K 16/2803; A61P 17/10

(57) ABSTRACT

Provided herein are methods for treating or preventing inflammatory skin disorders, such as Hidradenitis suppurativa, by administering to a subject having or at risk of developing the inflammatory skin disorder an agent that inhibits NK cell activity.

### CROSS REFERENCE TO RELATED APPLICATIONS

This application claims the benefit of U.S. Provisional Application No. 63/342,886, filed May 17, 2022, which is incorporated by reference herein in its entirety.

### STATEMENT REGARDING FEDERALLY SPONSORED RESEARCH

This invention was made with government support under ES026219, 5PO1CA210946, AI149267, and MDO15319 awarded by the National Institutes of Health. The government has certain rights in the invention.

### BACKGROUND

Hidradenitis suppurativa (HS) is a chronic debilitating inflammatory skin disease marked by persistent or recurrent papules, nodules, and abscesses (with or without sinus tracts) in the apocrine gland areas of the body, including the groin and axillary regions and under the breasts. Although the disease is underdiagnosed, currently, the estimated worldwide prevalence is 1-4% with a female-to-male ratio of 3:1. The mean onset of the disease is 21.8 years and individuals of African ancestry are more commonly affected than those of European ancestry. The reasons for the early onset, gender and racial differences in HS are unknown. The clinical features of the disease include painful nodules, malodorous discharge, recurrent abscesses and development of scars and fibrosis. The affected primary regions are the groin, axillae, and anal folds. Secondary affected regions are submammary skin tissue and skin folds particularly is obese individuals. The clinical features have a dramatically negative effect on the quality of life often contributing to depression and anxiety in the patients. Invasive squamous cell cancer (SCC) is considered one of the most severe complications of HS which carries a very high risk of death. HS is classified by Hurley stages I, II and III, based on disease severity and extent of affected area. Although inflammation is a key feature, major gaps in knowledge includes the mechanistic underpinning of the disease onset, progression, overall etiology, and the potential for targeted treatment.

The most common treatment for HS is systemic antibiotics and intralesional corticosteroids to treat infection and attenuate inflammation. TNF-α blockers are the only FDA approved biologics for the treatment of HS. Although anti-IL-17a (secukinumab) treatment has resulted in favorable clinical responses in treating HS in subjects with refractory disease and although targeting IL-17F and IL-1β for HS is currently in clinical trials, these treatments at best provide marginal relief. Furthermore, patients develop a resistance to one or more of these treats, manifesting refractory HS. Hence there is an essential unmet need for new treatment for HS.

### SUMMARY OF THE INVENTION

Provided herein is a method for treating or preventing an inflammatory skin disorder, such as HS, in a subject. The method comprises administering to a subject having or at risk of developing an inflammatory skin disorder an agent that inhibits NK cell activity. The agent that inhibits NK cell activity is optionally an agent that blocks the interaction of CD2 and CD58. The agent that blocks the interaction of CD2 and CD58 can be an anti-CD2 agent, such as an anti-CD2 antibody, or an anti-CD58 agent, such as an anti-CD58 antibody. The subject having the inflammatory skin disorder may have a refractory inflammatory skin disorder.

The methods provided herein optionally further comprising administering one or more additional therapeutic agents such as an antibiotic, a steroid, an anti-IL-15 agent, an anti-TNF-α agent, an anti-Il-17a agent, an anti-IL-18 agent, and/or an anti-fibrotic agent.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a schematic showing CD2 blockade attenuation of NKT and NK cell activities in HS pathogenesis. Subpopulations of NKT cells and NK cells drive tissue destruction and remodeling in HS. CD56bright NKT cell subpopulations expressing IFN-7, TGF-β, IL-13, IL-10, and/or IL-4 interact with keratinocytes and/or fibroblasts. CD11b+/−CD56dim NK cells producing IFN-7 interact with keratinocytes. Cognate interaction of CD2 on NKT and NK cell populations with CD58 on keratinocytes and fibroblasts is critical for NKT and NK cell dependent disease processes.

FIG. 2 shows analysis of scRNAseq quality control (QC) for 6 HS samples. Total molecules detected within each sample (nCount-RNA), total number of genes detected in each Sample (nFeature-RNA), and fraction of mitochondria genes (percent.MT) across recovered samples are shown before filter (FIG. 2A) and post filter (FIG. 2B). The data from four HS sample met QC and were used in subsequent analyses.

FIG. 3 shows transcriptomic analyses for identifying NK and NKT cell populations, the major innate immune cells in HS skin. FIG. 3A is a UMAP of scRNAseq transcriptomes from normal skin (n=4) and HS skin (n=4). Seurat 4.0 was used to identify 15 clusters representing immune and non-immune cell populations. FIG. 3B shows the cell type proportion in each cluster in normal and HS skin. FIG. 3C is a dot plot depicting normalized gene expression in HS for a panel of genes in each cell population. FIG. 3D is a volcano plot of differentially expressed genes in HS skin tissue (n=8) compared to normal skin tissue (n=6-8). Gene expression was determined using qRT-PCR OpenArray panels containing 2429 unique target genes. Significantly up regulated or down regulated genes are shown in the upper right or upper left, respectively (log 2FC≥2; P value≤0.05). FIG. 3E is a heatmap and FIG. 3F is a volcano plot of expressed miRNAs in normal and HS skin. miRNA profile from normal skin (n=6) and HS skin (n=9). Expression was determined using an OpenArray panel containing 754 well characterized miRNAs. The Volcano plot (FIG. 3F) shows differentially expressed miRNAs that were upregulated or downregulated in HS compared to normal skin (upregulated are shown on the upper right and down regulated are shown on the upper left. Eleven miRNAs with known functions in modulating NK cell differentiation and/or function are labeled in the volcano plot. FIG. 3G is a diagram depicting downstream gene targets of miRNA modulated in HS with relevant function in NKT and/or NK cells. Upregulated gene targets include Let-7, GZMB, PRF1, miR-29a-5p, miR-155, miR-21, miR-29b, Tbx21, miR-126, miR-142-3p, miR-150, miR-200a, and Stat4. Downregulated gene targets include PLZF, miR-27a-5p, miR-181a, CCND1/CDKN1A/GATA3, PTEN, IRS1 and c-Myb.

FIG. 4 provides an annotation of different cell types based on expression of various cell markers. FIG. 4A is a dot plot depicting normalized gene expression used for defining different cell populations. FIG. 4B and FIG. 4C shows cell population QC post filter based on number of molecules detected in each cell type (nCount-RNA) (FIG. 4B) and the number of genes detected within each cell type (nFeature-RNA) (FIG. 4C).

FIG. 5 shows the proportion of the cell populations in each of the 4 HS scRNAseq data used in downstream analysis.

FIG. 6 shows bulk transcriptomics analysis and qRT OpenArray PCR assay. FIG. 6A-6D are heatmaps representing bulk transcriptomic analysis of four publicly available datasets (GSE151243, GSE154773, GSE79150, and GSE128637) generated from both microarray and RNA-sequencing technologies. Data show comparison between controls (normal (N)/control/non-lesional) and HS (HS or lesioned skin). FIG. 6E is a dot plot depicting the significantly activated or inhibited canonical pathways in each dataset identified using Ingenuity Pathway Analysis (IPA) (−log (BH p-value≤0.05; z-score>|1|. Enriched pathways include T cell receptor signaling, Il-2 in activated T lymphocytes, and B cell development. FIG. 6F is a heatmap of qRT-PCR OpenArray analysis of four different signaling panels containing 2429 unique target genes in skin samples from normal (n=6-8) and HS (n=8) subjects. FIG. 6G is a bar graph showing activated cell signaling pathways in HS relative to normal based on log q-value.

FIG. 7A is a Venn diagram showing the use of HS tissue from patients in different experiments. Overlapping circles represent tissue used for more than one experiment. FIG. 7B is a scatter plot showing relative quantification of eleven miRNAs with known roles in modulating NKT/NK differentiation/maturation/function. Each dot represents tissue from an independent control (left) or HS (right) individual.

FIG. 8 shows spatial localization of NKT and NK cell populations in HS Skin. FIGS. 8A-8C show immunofluorescence staining for CD3, CD56 and CD2 expressing cells. FIGS. 8D-8F show immunofluorescence staining for CD2, CD58 and CD56 expressing cells. Images of normal or HS skin tissue are labeled. H&E staining shows “stitched skin” (center) with boxes denoting magnified areas of epidermis/hypodermis and sinus tract regions in HS. Scale bar of immunofluorescence images is 100 m. Arrows in the immunofluorescence images point to CD2+CD3+CD56bright (NKT cells) and CD2+CD3-CD56dim (mature NK cells) in the epidermal/hypodermal regions and sinus tract (FIG. 8A, FIG. 8C, and FIG. 8D). T cells in the immunofluorescence images are CD2+CD3brightCD56−. Immunofluorescent staining in the HS sinus tract (FIG. 8D) shows clusters of NKT cells that are spatially separated from NK cells. FIG. 8B, FIG. 8E and FIG. 8F show NK (CD2+CD56dim) and NKT (CD2+CD56bright) cells interacting with CD58 (keratinocyte or fibroblasts) expressing cells. FIG. 8B1, FIG. 8C1, FIG. 8C2, FIG. 8E1, FIG. 8F1 and FIG. 8F2 show zoomed in regions of cell populations in greater detail. Data are representative of HS skin (n=7-16) and normal skin (n=minimum 3) sections for immunofluorescence studies. H& E staining was carried out for all the HS and normal skin sections.

FIG. 9 are H&E stained micrographs of normal (n=3) and HS (n=3) skin tissues. The images show morphological anomalies in HS skin such as epidermal hyperplasia, leukocytes infiltration and tunnels. (Magnification=4×, Scale bars=200 m and 300 M for stitched image).

FIG. 10 are micrographs of immunofluorescence staining for each individual marker. (Resolution 4096×4096, Magnification=×20, Scale bars=100 m).

FIG. 11 are micrographs of immunofluorescence staining for each individual marker. (Resolution 4096×4096, Magnification=×20, Scale bars=100 m).

FIG. 12 shows micrographs of immunofluorescence staining for CD8 T cells in HS. (Resolution 4096×4096, Magnification=×20, Scale bars=100 m).

FIG. 13 shows micrographs of H&E stained skin sections (center) and immunofluorescence staining for CD4 T cells, CD68 and CD20 B cells in HS. FIGS. 13A-13C show CD4 and CD68 expressing cells in normal and HS skin. FIGS. 13B-13F show CD20 expressing cells in normal and HS skin. Arrows point to CD20+ cells. FIGS. 13G-13H are micrographs of immunofluorescence staining for each individual marker shown in FIGS. 13A-13C & FIGS. 13D-13F. (Resolution 4096×4096, Magnification=×20, Scale bars=100 m).

FIG. 14 are micrographs of immunofluorescent stained plasma cells in HS. for each individual marker (CD2, CD3 and IgG. (Resolution 4096×4096, Magnification=×20, Scale bars=100 m).

FIG. 15 shows micrographs of immunofluorescence staining for each individual marker (DAPI, CD56, CD3, Perforin-1, and CD2) in Normal and HS skin samples. (Resolution 4096×4096, Magnification=×20, Scale bars=100 m).

FIG. 16 shows micrographs of immunofluorescence staining for each individual marker, (DAPI, CD2, GZMA, CD56, and CD3) in Normal and HS skin samples. (Resolution 4096×4096, Magnification=×20, Scale bars=100 m).

FIG. 17 shows micrographs of immunofluorescence staining for each individual marker, (DAPI, CD56, CD2, CD11, and GZMB). (Resolution 4096×4096, Magnification=×20, Scale bars=100 m).

FIG. 18A is a UMAP of NKT and NK cell clusters in HS extracted from scRNASeq analysis from FIG. 3A. FIG. 18B shows the cytotoxicity score of NKT and NK cells from HS Samples. FIG. 18C is a UMAP dimensional reduction clustering identifying six different sub-clusters of NKT (clusters 0-2), NK (clusters 3 and 5) and MAIT (cluster 4) cells. FIG. 18D shows expression of NKT and NK associated genes in each of the six sub-clusters. The analysis shows that populations 0, 1, 2 and 4 express CD3. Population 4 has the gene expression profile of MAIT cells. Populations 3 and 5 have gene expression profiles of NK cells. FIG. 18E shows the proportion of NKT, NK and MAIT cell populations in scRNAseq data from each of the four HS patients.

FIG. 19 shows scored for inflammation (FIG. 19A) and cytotoxicity (FIG. 19B) in different NKT and NK cell sub-clusters from individual HS Samples. NK populations had similar inflammation scores, whereas population 3 (NK) and population 4 (MAIT) had the highest cytotoxicity scores.

FIG. 20 shows bar graphs of expression of cytokines, chemokines and growth factors involved in inflammation, recruitment of effector immune cells, and cell proliferation in normal (n=11) (left) and HS (n=13) (right) skin samples. Two tailed Student's t-Test of normal vs HS (*P≤0.05, **P≤0.01; ***P≤0.001; ****P≤0.0001).

FIG. 21 are graphs showing the levels of cytokines, chemokines, and growth factors in normal (n=11) and HS (n=13) skin samples. The data represent analytes in which HS was not significantly different from normal. Data are from the multiplex Luminex platform using 45-Plex Human ProcartaPlex™ Panel 1 (Cat #EPX450-12171-901, ThermoFisher, Waltham, MA).

FIG. 22 shows that CD2 blockade in organotypic HS skin culture attenuates proinflammatory and fibrotic signaling pathways. FIGS. 22A and 22B are volcano plots depicting changes in gene expression induced by CD2 blockade as determined by TaqMan based human inflammatory OpenArray panel. The data show that CD2 blockade in HS skin organotypic cultures down regulates the expression of several inflammatory and fibrotic gene markers at the transcriptional level. The same three skin tissue were used for control-IgG and anti-CD2 treatment.

FIG. 23 are graphs showing the levels of cytokine, chemokine, and growth factor stimulation in organotypic cultures of HS skin treated with anti-CD2 or IgG.

FIG. 24 are graphs showing the levels of cytokine, chemokine, and growth factor stimulation in organotypic cultures of HS skin treated with anti-CD2 or IgG along with LPS.

FIG. 25 shows upstream regulators as determined by IPA analysis. Upstream regulators of HS compared to normal OpenArray data from FIG. 3D. FIG. 25A shows the top four significantly upregulated upstream regulators namely, lipopolysaccharide (P≤8.27E-81, z-score=6.550), TNF (P≤6.13E-70, z-score=4.994), IFNG (P≤3.96E-64, zscore=5.459) and TGFB1 (P≤1.12E-60, z-score=3.963) in untreated lesioned HS skin versus healthy controls. FIG. 25B shows that the upstream regulators TNF (P≤1.70E-32, z-score=−3.721), lipopolysaccharide (P≤1.61E-28; z-score=−4.288), IFNG (P≤1.72E-27; z-score=−2.993) and TGFB1 (P≤3.52E-26, z-score=−2.236) were significantly downregulated in HS skin after being treated with anti-CD2 compared to IgG treated HS skin in organotypic cultures.

FIG. 26 shows bar graphs of significantly activated or inhibited signaling pathways using IPA analysis of HS versus normal skin samples (FIG. 26A) and anti-CD2 versus control IgG treated HS skin samples (FIG. 26B).

FIG. 27 shows a summary of the IPA analysis of HS versus normal skin samples (FIG. 27A) and anti-CD2 versus control IgG treated HS skin samples.

### DETAILED DESCRIPTION

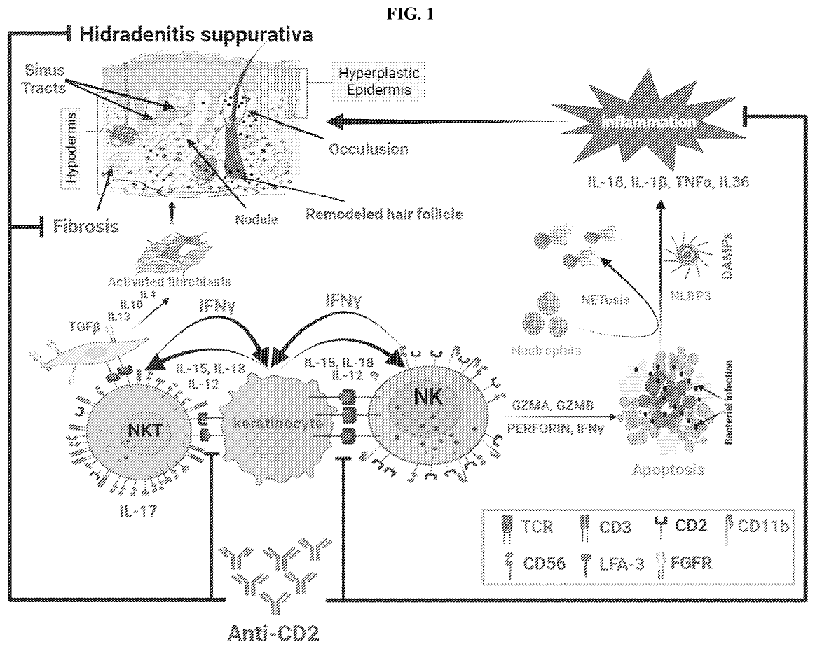

A novel role for innate immune lymphocytes, specifically NKT cells and NK cells, in HS pathogenesis is identified herein. In particular, a striking heterogeneity in NKT and NK cell populations in HS skin is identified, with each subpopulation contributing to nonoverlapping aspects of disease pathogenesis as depicted in FIG. 1. A common feature of all NKT and NK cell populations was the elevated expression of CD2, an adhesion and activating receptor, which is integrally involved in the cellular network of HS pathogenesis through its interaction with CD58 on keratinocytes and fibroblasts. Disruption of this interaction using an anti-CD2 blocking antibody attenuated HS pathogenesis associated cytokine/chemokine protein and gene expression. Importantly, the HS tissues studied here were mostly from patients who were refractory to all previous treatments. Therefore, the striking contribution of NK cell populations reflects a novel therapeutic intervention for all stages of HS disease, including HS refractory to current treatments.

The major immune cell populations implicated to date in HS have been T cells, B cell populations (B cells and plasma cells), neutrophils, and macrophages. Th1 and the CD8+ T cell cytokines, IFN-7 and TNF-α, are highly elevated in HS. Similarly, the Th17 signature is prominent in HS, including IL-17A, IL-17F, IL-22 and IL-6. Further, neutrophils are recruited into HS lesions perhaps mediated by IL-17, IL-8 and/or infection. Neutrophils form neutrophil extracellular traps (NETs) within HS lesions promoting in situ immune dysregulation and amplification of the inflammatory cascade through activation of the NLRP3-inflammasome pathway and/or suppression of immune regulatory mechanisms. The inflammatory milieu of HS also recruits monocytes and promote their differentiation into inflammatory MI-like macrophages.

Recruitment and activation of innate and adaptive immune cells around hair follicles drive initiation and progression of HS. However, the nature of the cell populations and underlying mechanisms remain unclear. Analysis of HS and normal skin by scRNAseq revealed that natural killer (NK) cells and CD4 T cells were the major lymphocyte populations in HS. Confocal scanning showed that HS, but not normal skin, contained elevated numbers of CD56^{dim} and CD56^{bright} NK cells. The epidermis and sinus tracts regions of HS primarily contained CD56^{dim} NK cells, that expressed high levels of perforin and granzyme A and no CD3, a population representing highly cytolytic classical NK cells. In contrast, sinus tracts were enriched in CD56^{bright} NK cells that expressed CD3, a population defining NK-T cells. These NK-T cells were juxtaposed with α-SMA expressing fibroblasts, a feature of fibrosis. Notably, CD56^{dim} NK cells expressed high levels of CD2, a receptor that signals augmented NK cell cytolytic activity and increased production of IFN-γ. These NK cells associated with keratinocytes expressing CD58 (LFA-3), the counter-receptor for CD2. The keratinocytes contained significantly elevated levels IL-15 and IL-18, cytokines induced after engagement of CD58 with CD2. HS skin also expressed elevated levels of miR150 and miR155, micro RNAs necessary for the NK cell maturation and cytotoxicity, respectively. To test that CD2 blockade in HS leads to reduction in HS associated chemokines and cytokines, skin from HS patients were cultured in presence of anti-CD2 blocking Ab or control IgG. CD2 blockade led to significant decrease in inflammatory cytokines (IL-6, IL-15, IL-18, IFN-γ) and chemokines (IL-8, MIP1α, RANTES, IP10). This study showed that distinct NK cell populations are the major contributors to cytolytic activity as well as fibrosis in HS. Thus, using a multi-omics approach including single-cell and bulk transcriptomes and regulomes to build deterministic associations among multiple biological factors in HS, NK cell populations, CD3+CD56bright NKT cells and classical cytolytic CD56dim NK cells were identified as the major innate lymphoid cells contributing to HS disease. These NKT cells and NK cells were partitioned at different locations within HS skin contributing to different aspects of HS pathogenesis. Molecularly, the NKT and NK cells expressed high levels of the co-stimulation/adhesion molecule CD2, and its blockade attenuated the inflammatory gene and cytokine expression profile of HS. Thus, CD2 blockade is identified as a new therapeutic approach to the treatment of HS.

Provided herein are methods for treating or preventing inflammatory skin disorders, such as, but not limited to, HS, cutaneous Crohn's disease, pyoderma gangrenosum, Sweet's syndrome, and psoriasis. The methods comprise administering to a subject having the inflammatory skin disorder or at risk of developing the inflammatory skin disorder an agent that inhibits NK cell activity. In some embodiments, the skin disorder is associated with increased NK cells and/or increased NK cell cytolytic activity.

In some embodiments, the agent that inhibits NK cell activity is an anti-CD2 agent, an anti-IL15 agent, and/or an anti-IL-18 agent. Optionally, agent that inhibits NK cell activity is an agent that blocks the interaction of CD2 and CD58. In some methods, the agent is an antibody (e.g., an anti-CD2 antibody or an anti-CD58 antibody) or a small molecule. The antibody is optionally a humanized antibody or a portion thereof. By way of example Suplizumab is a humanized monoclonal anti-CD2 IgG1 antibody useful in the methods described herein. Effective fragments of Suplizumab or other whole antibodies can be used.

The agent that inhibits NK cell activity is optionally a small molecule or a blocking peptide that inhibits CD2 signals or activity.

In addition to agents that inhibit NK cell activity, also useful in the methods described herein are agents that indirectly inhibit NK cell activity by affecting upstream or downstream functions.

As used herein, the term antibody includes intact polyclonal or monoclonal antibodies, single chain antibodies, or antibody fragments, such as Fab, Fab′, and F(ab′)2, scFv, Fv, dsFv diabody, and Fd fragments as well as combinations of such antibodies or fragments. Thus, provided herein are intact polyclonal or monoclonal antibodies, single chain antibodies, or antibody fragments, such as Fab, Fab′, and F(ab′)2, scFv, Fv, dsFv diabody, and Fd fragments that bind CD2, CD58, or the like. Such antibodies or fragments optionally demonstrate the same epitope specificity as Suplizumab,

The term antibody also includes chimeric antibodies (e.g., humanized antibodies) and hybrid antibodies, with dual or multiple antigen or epitope specificities. Thus, fragments of the antibodies that retain the ability to bind their specific antigens (e.g., CD2m CD58, etc.) are provided. Also included within the meaning of antibody or fragments thereof are conjugates of antibody fragments and antigen binding proteins (single chain antibodies).

As described herein, inhibition of NK cell activity includes any detectable decrease as compared to normal (non-disease) sample, such as a normal skin sample. Such a decrease can be at least 10%, 20%, 30%, 40%, 50%, 60%, 70%, 80%, 90% or 100% as compared to the control level. NK cell activity can be assessed using a variety of methods, including without limitation ⁵¹Cr-release assays or flow cytometry-based assays revealing the cells' cytotoxic capacity or by stimulating them to produce cytokines. Various methods are described in the Examples below. Thus, an effective amount of agent that inhibits NK cell activity can be detected directly by such methods or an effective amount can be determined based on clinical improvement, for example, reduced pain, reduced skin lesions, or the like.

Agents that inhibit NK cell activity can be administered to subjects at risk for developing inflammatory skin disorders as described here. Such subjects include those with inflammatory disorders including gastrointestinal diseases (like inflammatory bowel diseases such as Crohn's Disease), arthritis, acne, diabetes, and metabolic syndrome. Subjects at risk can also include those with a family history of inflammatory diseases. Additionally, chronic smoking and obesity are risk factors for the development of inflammatory skin disorders like HS.

Provided herein are methods for treating or preventing inflammatory skin disorders in a subject by administering to the subject having the inflammatory skin disorder or at risk of developing the inflammatory skin disorder an agent that inhibits NK cell activity, as described herein, and an additional therapeutic agent. Additional therapeutic agents can be selected from the group consisting of biologics (e.g., infliximab and adalimumab), antibiotics, steroids, TNF-α blocker, anti-IL-17a (e.g., secukinumab), hormone supplements, retinoids, metformin, anti-fibrotic agent, and pain medications. Such additional agents can be administered prior to, following, or concurrent with treatment with the agent that inhibits NK cell activity. The agent that inhibits NK cell activity is optionally administered either concomitantly (e.g., as an admixture), separately but simultaneously (e.g., via separate intravenous lines into the same subject), or sequentially (e.g., one of the compounds or agents is given first followed by the second) with the additional therapeutic agent. Thus, the term combination is used to refer to concomitant, simultaneous, or sequential administration of two or more agents.

Optionally, the subject to be treated has an inflammatory skin disorder that is refractory to one or more previous treatments of the inflammatory skin disorder, wherein the previous treatment was not treatment with an agent that blocks the interaction of CD2 and CD58. The treatment to which the subject is refractory includes, for example, antibiotics, corticosteroids, TNF-α blockers, anti-IL-17a (e.g., secukinumab) treatment, and the like. In a subject refractory to the previous treatment, however, the agent used in the previous treatment can be used concomitantly, simultaneously, or sequentially with an agent that blocks the interaction of CD2 and CD58.

Pharmaceutical compositions comprising an agent that inhibit NK cell activity are provided herein. A pharmaceutical composition can include a therapeutically effective amount of any compound described herein. In some embodiments, the pharmaceutical composition can further comprise a carrier. Such effective amounts can be readily determined by one of ordinary skill in the art as described above. Considerations include the effect of the administered compound, i.e., agent that inhibits NK cell activity, or the combinatorial effect of the compound with one or more additional active agents, if more than one agent is used in or with the pharmaceutical composition. Cell culture assays and animal studies can be used in formulating a range of dosage for use in humans.

The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water. Carriers can include any excipient, diluent, filler, salt, buffer, stabilizer, solubilizer, lipid, stabilizer, or other material known in the art for use in pharmaceutical formulations.

Depending on the intended mode of administration, the pharmaceutical composition can be in the form of solid, semi-solid or liquid dosage forms, such as, for example, tablets, suppositories, pills, capsules, powders, liquids, lotions, or suspensions, optionally in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained. The composition can also include additional agents, such as wetting, emulsifying, suspending, sweetening, flavoring, or perfuming agents.

The agents or compositions described herein can be administered in a number of ways depending on whether local (e.g., topical) or systemic treatment is desired, and on the area to be treated. The compositions are administered via any of several routes of administration, including orally, parenterally, intravenously, intraperitoneally, intraventricularly, intramuscularly, subcutaneously, intracavity or transdermally. Pharmaceutical compositions can also be delivered locally to the area in need of treatment, for example by topical application or local injection. Effective doses for any of the administration methods described herein can be extrapolated from dose-response curves derived from in vitro or animal model test systems.

Regardless of the route of administration, the amount of the reagent administered or the schedule for administration will vary among individuals based on age, size, weight, condition to be treated, mode of administration, and the severity of the condition. One skilled in the art will realize that dosages are best optimized by the practicing physician and methods for determining dosage are described, for example in Remington's Pharmaceutical Science, latest edition. A typical dose of the antibody used alone might range from about 1 μg/kg to up to 100 mg/kg of body weight or more per day, and preferably 1 μg/kg to up to 1 mg/kg, depending on the factors mentioned above. An intravenous injection of the antibody or fragment thereof, for example, could be 10 ng-Ig of antibody or fragment thereof, and preferably 10 ng-1 mg depending on the factors mentioned above. For local injection, a typical quantity of antibody ranges from 1 pg to 1 mg. Preferably, the local injection would be at an antibody concentration of 1-100 μg/ml, and preferably 1-20 μg/ml.

As used herein the terms treatment, treat, or treating refers to a method of reducing one or more of the effects of the disorder or one or more symptoms of the disorder, for example, an inflammatory skin disorder in the subject. Thus, in the disclosed methods, treatment can refer to a 10%, 20%, 30%, 40%, 50%, 60%, 70%, 80%, 90%, or 100% reduction in the severity of the inflammatory skin disorder. For example, a method for treating HS is considered to be a treatment if there is a 10% reduction in one or more symptoms of the HS in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

As utilized herein, by prevent, preventing, or prevention is meant a method of precluding, delaying, averting, obviating, forestalling, stopping, or hindering the onset, incidence, severity, or recurrence of a disease or disorder. For example, the disclosed method is considered to be a prevention if there is a reduction or delay in onset, incidence, severity, or recurrence of the inflammatory skin disorder or one or more symptoms of an inflammatory skin disorder in a subject susceptible to an inflammatory skin disorder as compared to control subjects susceptible to an inflammatory skin disorder that did not receive a composition described herein The disclosed method is also considered to be a prevention if there is a reduction or delay in onset, incidence, severity, or recurrence of an inflammatory skin disorder or one or more symptoms of an inflammatory skin disorder in a subject susceptible to an inflammatory skin disorder after receiving a composition described herein as compared to the subject's progression prior to receiving treatment. Thus, the reduction or delay in onset, incidence, severity, or recurrence of the inflammatory skin disorder can be about a 10, 20, 30, 40, 50, 60, 70, 80, 90, 100%, or any amount of reduction in between.

As used herein, administer or administration refers to the act of introducing, injecting or otherwise physically delivering a substance as it exists outside the body (e.g., an agent that inhibits NK cell activity) into a subject, such as by topical, mucosal, intradermal, intravenous, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

As used herein, the term therapeutically effective amount or effective amount refers to an amount of a compound or composition described herein, that, when administered to a subject, is effective to treat a disease or disorder either by one dose or over the course of multiple doses. A suitable dose can depend on a variety of factors including the particular agent used and whether it is used concomitantly with other therapeutic agents. Other factors affecting the dose administered to the subject include, e.g., the type or severity of the disease. For example, a subject having HS may require administration of a different dosage of a composition comprising an agent that inhibits NK cell activity as compared to subject with psoriasis.

A therapeutically effective amount is also one in which any toxic or detrimental effects of the composition are outweighed by the therapeutically beneficial effects.

As used in this specification and the appended claims, the singular forms a, an, and the include plural reference unless the context clearly dictates otherwise.

The terms may, may be, can, and can be, and related terms are intended to convey that the subject matter involved is optional (that is, the subject matter is present in some examples and is not present in other examples), not a reference to a capability of the subject matter or to a probability, unless the context clearly indicates otherwise.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The following examples revealed a striking heterogeneity in NKT and NK cell populations in HS skin, with each subpopulation contributing to nonoverlapping aspects of disease pathogenesis as depicted in FIG. 1. A common feature of all NKT and NK cell populations is the elevated expression of CD2, an adhesion and activating receptor, which is integrally involved in the cellular network of HS pathogenesis through its interaction with CD58 on keratinocytes and fibroblasts. Anti-CD2 treatment (referred to as a CD2 blockade) disrupts this interaction by blocking CD2-CD58 (LFA-3) binding and attenuating HS pathogenesis associated cytokine, chemokine, protein, and gene expression. Thus, the CD2 blockade is a highly potent suppressor of the immunopathogenesis of HS disease. Importantly, the HS tissues studied here are mostly from patients who were refractory to all previous treatments. Therefore, the striking contribution of NK cell populations reflect a novel therapeutic intervention for such a patient population.

### Example 1: Single Cell Preparation

In compliance with the protocol approved by the Institutional Review Board of the University of Alabama at Birmingham, surgically discarded skin tissues from heathy and HS (Hurley II/III stage) subjects were used. The surgically removed fresh human tissue (3×3 cm) was placed in 5 ml of Medium 154 (Thermo Fisher (Waltham, MA)) supplemented with human keratinocyte growth supplement and further cut into small pieces. 100 μl of freshly prepared Liberase TL (2 mg/mL stock, Roche Diagnostics, Indianapolis, IN) and 700 μl of Tyrode's solution were added to sterile Petri-dishes and tissues were gently minced using 15 ml centrifuge tube followed by incubation at 37° C. on water bath for 15 minutes. The suspension mixture was passed through a 70 m cell strainer (Falcon). Finally, any remaining tissue was incubated in growth medium supplemented with a solution containing 100 μl of 0.25% trypsin and 700 μl of Tyrode's solution and strained through the 70 μm cell strainer into the previous batch of cells. Cells were then re-suspended in 300-500 μl of sterile PBS with 0.04% BSA in a fresh 1.5 ml Eppendorf tube and centrifuged at 2000 rpm for 5 minutes. Resuspension and centrifugation steps were repeated before checking cell viability using 7-AAD Cells.

### Example 2: Single Cell RNA Sequencing Analysis and Clustering of HS Samples

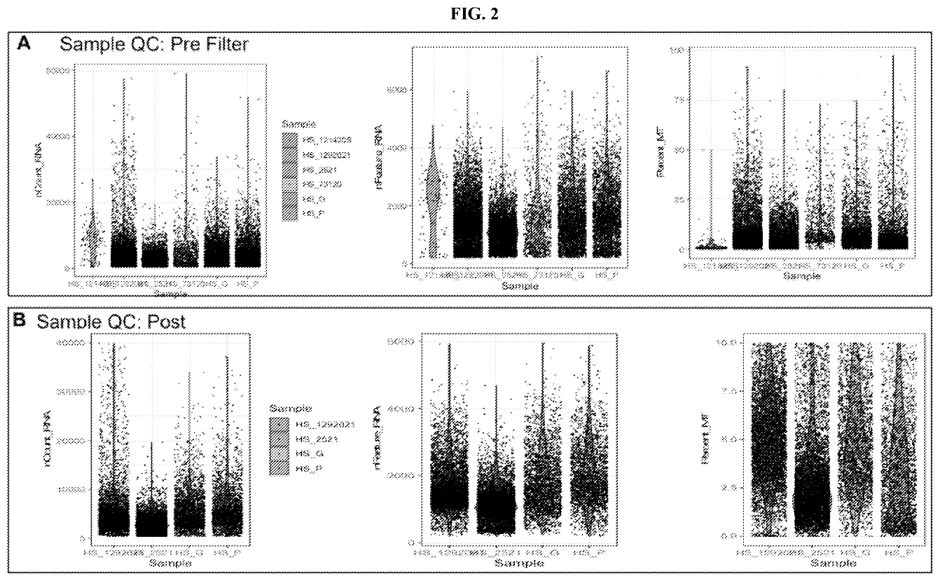

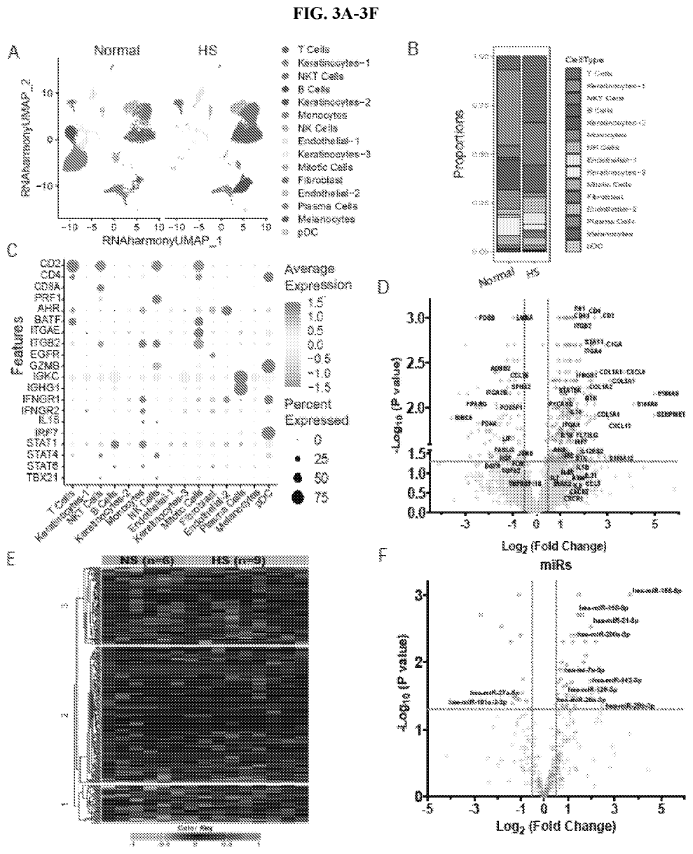

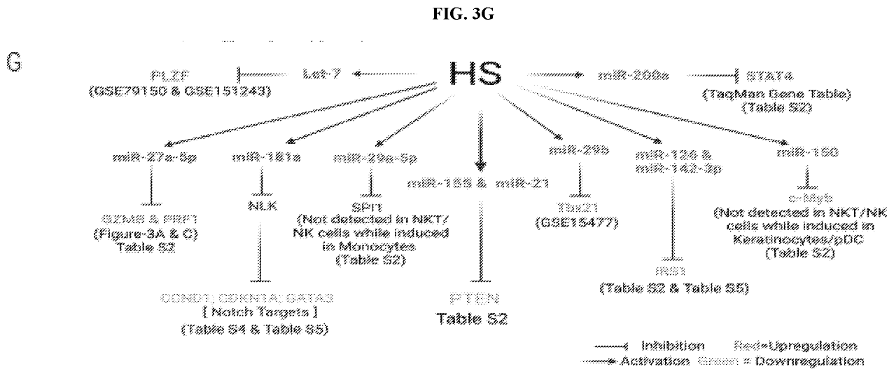

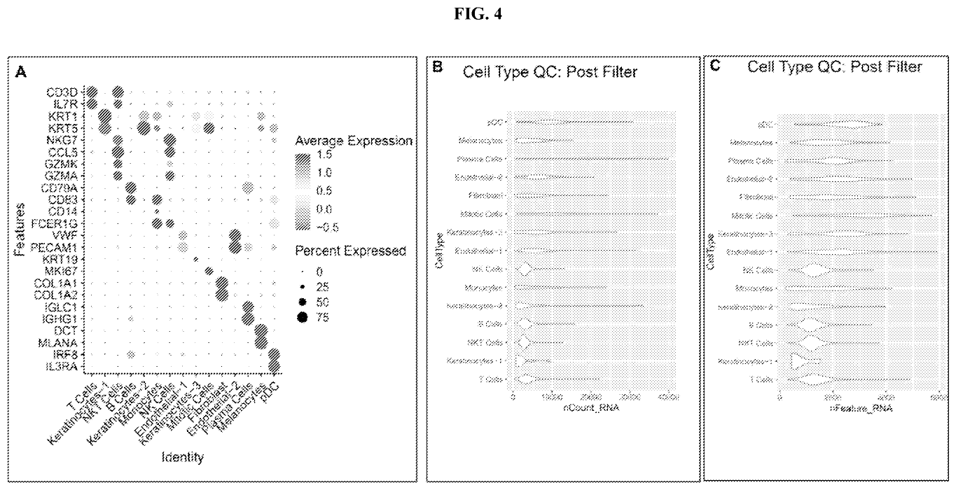

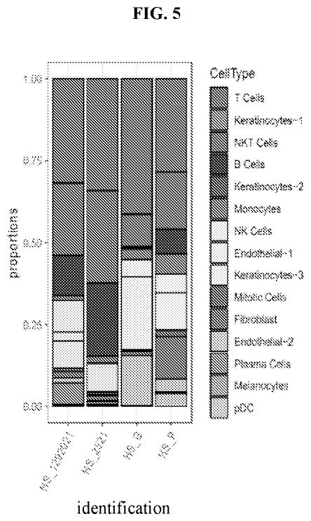

To identify immune cell populations associated with HS, single-cell RNA-seq (scRNAseq) using 10× Genomics protocols was performed. For scRNAseq analysis, normal skin data were from one in-house and three publicly available (GSM4284228, GSM4284235, and GSM4284237) NCBI GEO listed dataset (GSE144236). The HS skin data were obtained from six HS patients (Table 1), of which four patients met quality control requirements (FIGS. 2A and 2B). Data processing included quality control, read alignment, and gene quantification, which was done using the 10× Genomics Cell Ranger (v.5.0.1) tool (10× Gebnomics, Pleasanton, CA). For normalization, the Seurat (v.4.0) package was used. During quality control, some ribosomal protein genes and the over expressed MALAT1 gene were removed. Doublets/multiples of cells and doublets were removed using the Doublet Finder package. The Harmony package was used for data integration and samples were clustered using Seurat with 25 dimensions and 0.3 cluster resolution for all downstream analysis. Azimuth based markers and HS scRNAseq cell type identification were used to annotate cell populations. (See, Gudjonsson J E, Tsoi L C, Ma F, Billi A C, van Straalen K R, Vossen A, et al., JCI Insight. 2020; 5(19)). A dimensional reduction algorithm identified 15 clusters comprising populations of innate and adaptive immune cells, keratinocytes, fibroblasts, endothelial cells, and melanocytes (FIG. 3A and FIGS. 4A-4C). The major lymphoid cell populations identified were T cells, B cells, NKT cells, and NK cells (FIG. 3A and FIG. 5). The expanded proportion of NKT cells and NK cells in HS is a novel finding (FIG. 3B); and was present in each of the four HS scRNAseq data (FIG. 5 and Table 2).

TABLE 1
Patient (n = 30) and Control (n = 15) Demographics

| | Group | Ethnicity | Position | Gender |
|---|---|---|---|---|
| 1 | Patient Skin # 1 | Black | R-Breast (B) | F |
| 2 | Patient Skin # 2 | Black | Armpit (P) | F |
| 3 | Patient Skin # 3 | Black | Gluteal (O) | F |
| 4 | Patient Skin # 4 | Black | Groin (G) | F |
| 5 | Patient Skin # 5 | Black | Groin | F |
| 6 | Patient Skin # 6 | Black | Back (O-b) | F |
| 7 | Patient Skin # 7 | Black | Armpit (P) | F |
| 8 | Patient Skin # 8 | Black | Abdominal (A) | F |
| 9 | Patient Skin # 9 | Unknown | right-axilla | F |
| 10 | Patient Skin # 10 | Black | Axilla (P) | F |
| 11 | Patient Skin # 11 | Black | Axilla (P) | F |
| 12 | Patient Skin # 12 | Black | Brest (B) | M |
| 13 | Patient Skin # 13 | Black | Armpit(P) | F |
| 14 | Patient Skin # 14 | Black | sacral area/thigh | F |
| 15 | Patient Skin # 15 | Black | R-Groin | F |
| 16 | Patient Skin # 17 | Black | Armpit(P) | F |
| 17 | Patient Skin # 19 | Black | Axilla (P) | M |
| 18 | Patient Skin # 22 | Black | L-Buttock (O) | M |
| 19 | Patient Skin # 28 | Black | arm | F |
| 20 | Patient Skin # 29 | Black | axilla = Armpit (P) | F |
| 21 | Patient Skin # 30 | Black | Unknown | |
| 1 | Normal Skin #1 | Caucasian | L-Breast | F |
| 2 | Normal Skin #2 | Black | L-Armpit (P) | F |
| 3 | Normal Skin #3 | Black | Back (O) | M |
| 4 | Normal Skin #4 | Black | Breast (B) | F |
| 5 | Normal Skin #5 | Black | Abdominal | M |
| 6 | Normal Skin #6 | Black | Breast (B) | F |
| 7 | Normal Skin #7 | Black | Breast (B) | F |
| 8 | Normal Skin #8 | Black | Breast (B) | F |
| 9 | Normal Skin #9 | Caucasian | Breast (B) | F |
| 10 | Normal Skin #10 | Black | Breast (B) | F |
| 11 | Normal Skin #11 | Black | Unknown | M |
| 12 | Normal Skin #22 | Unknown | Abdominal (A) | M |
| 13 | Normal Skin #26 | Black | Breast (B) | F |
| 14 | Normal Skin #27 | Black | Breast (B) | F |
| 15 | Normal Skin #28 | Black | R-Breast (B) | F |

TABLE 2
Absolute Number of Cells Captured per Cell Population from Each HS Patient

| Cell Types | Control BA | Patient # 4 | Patient # 7 | Patient # 10 | Patient # 11 |
|---|---|---|---|---|---|
| T Cells | 380 | 1564 | 1950 | 940 | 615 |
| Keratinocytes-1 | 0 | 8 | 0 | 3 | 0 |
| NKT Cells | 539 | 1082 | 1608 | 225 | 378 |
| B Cells | 7 | 607 | 1278 | 9 | 162 |
| Keratinocytes-2 | 0 | 2 | 5 | 7 | 1 |
| Monocytes | 18 | 72 | 120 | 75 | 133 |
| NK Cells | 249 | 476 | 494 | 119 | 124 |
| Endothelial-1 | 58 | 131 | 20 | 508 | 243 |
| Keratinocytes-3 | 3 | 409 | 33 | 5 | 4 |
| Mitotic Cells | 3 | 57 | 16 | 6 | 42 |
| Fibroblast | 51 | 95 | 88 | 32 | 280 |
| Endothelial-2 | 10 | 76 | 13 | 348 | 86 |
| Plasma Cells | 0 | 324 | 56 | 1 | 8 |
| Melanocytes | 2 | 20 | 25 | 0 | 2 |
| pDC | 2 | 7 | 8 | 3 | 85 |

Several genes associated with lineage and/or function were prominently expressed within the immune cell populations from HS samples (FIG. 3C and FIG. 4A). CD2, a cell surface receptor associated with NKT cells, was highly expressed in NIK and T cells in HS (FIG. 3C). Cells within the T cell cluster predominantly expressed CD4 rather than CD8A or CD8B, indicating that they were helper T (Th) cells rather than cytotoxic T cells ( ). CD8A expression is also observed within the NK cell cluster, a feature consistent for some NTT cells. These CD8A expressing NKT cells were unlikely to be misclassified CD8 T cells as they did not express significant levels of BATF, ITGAE or ITGB2 transcription factors. Other genes identified with elevated expression in NKT and NK cells and associated with enhancing their maturation/activation were NCAM1 (CD56), PRF1, TBX21, STAT1, STAT4, PDCD2, GZMA, GZMB, and the cytokine IFNG (FIGS. 3C and 4A). Genes with decreased expression in HS NKT and NK cells were PRDX2, STAT6, IFNAR1. IFNGR1 and IFNGR2 (FIG. 3C). T cells in HS expressed CD4, STAT1, STAT4, RORC, PDCD1, PDCD2, FOXP3, BATF, AHR, and the cytokines IFNG, IL-17A, IL-17F, and IL-21 (FIG. 3C). The expression of these genes in T cells are associated with effectors (Th1 and Th17) and regulatory (Treg) phenotypes. STAT6, the transcription factor induced and activated by the type 2 cytokine IL-4, was significantly reduced in HS T cells. This indicates that type 2 immune response lymphoid cells (Th2 or ILC2) are underrepresented in HS. PDCD2, AHR, BATF, and STAT1 were increased in a small percent of HS resident B cells and plasma cells (FIG. 3C). CD8 T cells contribute minimally to HS. The monocyte/macrophage cell cluster exhibited an inflammatory gene signature with elevated expression of STAT1, IFNGR1, IFNGR2, IRF7 (FIG. 3C), genes associated with activation of type 1 and type II IFN signaling (FIG. 3C). This cluster along with keratinocytes has enhanced expression of IL-18, a cytokine that promotes activation and expression of IFN-7 in T cells and NK cells. Overall, the data indicate that the major lymphocyte populations present in HS skin are NK cells, CD4 T cells, and B lineage cells.

### Example 3: Bulk Transcriptomics Data Acquisition and Analysis

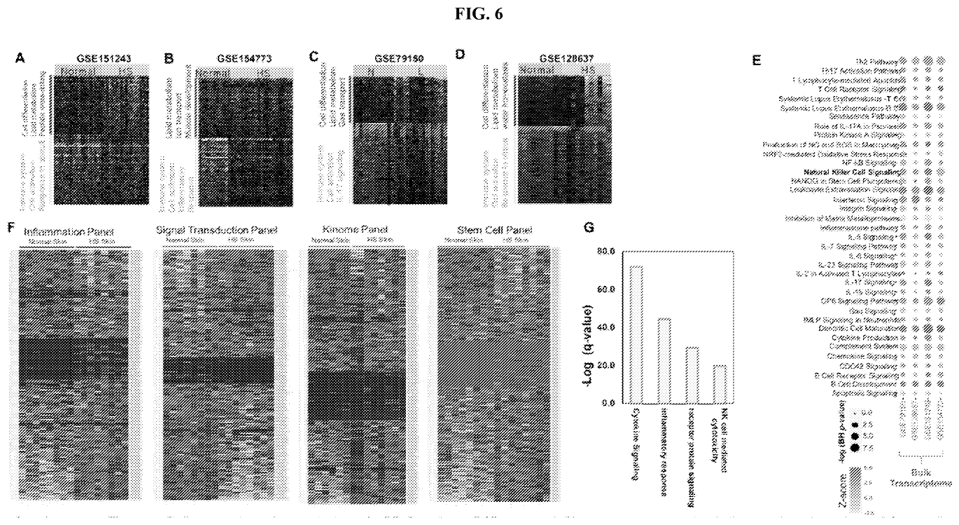

To study the bulk transcriptome expression profiles altered by HS, four publicly available datasets (GSE151243, GSE154773, GSE79150, and GSE128637) representing both microarray and RNA-sequencing technologies from NCBI GEO database were used. Raw values were log 2 normalized for further analyses hereafter. DESeq2 was used to identify differentially expressed genes (DEGs) with default parameters (FDR<0.05 and log 2FC=|1|) independently. For each HS sample, 1614, 2080, 434 and 359 significantly up-regulated genes and 1354, 1747, 309 and 300 down regulated genes were identified as compared to control, respectively (FIGS. 6A-6D). Ingenuity Pathway Analysis (IPA) revealed that NK cell signaling, and other pathways are significantly activated in HS (FIG. 6E).

### Example 4: Quantitative PCR Open Arrays Analysis

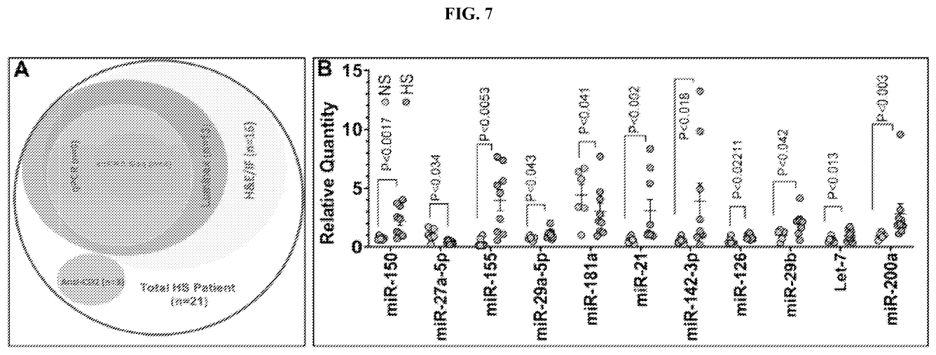

To validate global differential gene expression from publicly available RNASeq datasets of normal (n=6-8) and HS (n=8) skin samples, quantitative TaqMan based real-time qPCR for predesigned gene panels of various regulatory pathways was employed. Of the 8 HS tissues analyzed in this assay, four were the same individuals used for scRNAseq (FIG. 7A). The human Inflammation open array panel (Cat #4475389), human signal transduction panel (Cat #4475392), human kinome panel (Cat #4475388) and human stem cell openarray panel (Cat #4475390, Thermo Fisher, USA) were used. Briefly, total RNA from HS and normal skin tissue was isolated using Trizol reagent (Cat #15596018, Ambion (Thermo Fisher). A total of 2 g of RNA was reverse transcribed into cDNA using the SuperScript® VILO™ cDNA Synthesis Kit (Cat #11754250, Life Technologies (Thermo Fisher)). Pre-amplification of cDNA was performed in 25 μl total volume containing 12.5 μl of TaqMan preamp master mixture (Cat #4391128), 2.5 μl custom TaqMan preamp primers pools (Cat #4441856, ThermoFisher), 2.5 μl reverse transcription product. Pre-amplification thermal cycling conditions included incubation of products at 95° C. for 10 min followed by 12× cycles at 95° C. for 15 seconds and 4 minutes at 60° C. The pre-amplification products obtained after the cycling were incubated for 10 minutes at 99.9° C. and then diluted 20-fold and used in final implication reaction as per the manufacturer's instructions. OpenArray chips that altogether contained the pre-coated primers for the 2429 targets were read on the 12K Flex RT-PCR machine (Thermo Fisher Scientific). Data analysis was performed through the online available Expression suit (v 1.3) software (Thermo Fisher Scientific) using global gene normalization method.

### Example 5: qRT-PCR Analysis of Gene Expression

From the qRT-PCR OpenArrays representing 2429 gene targets, 170 genes were significantly (Log 2FC≥1; p-value<0.05) increased and 66 genes were significantly (Log 2FC≤−1; p-value<0.05) decreased in HS compared to controls (FIG. 3D and FIG. 6F). Those genes whose expression was significantly elevated in HS compared to controls include CD2, kinase BTK, integrin ITGAX, complement protein C1QA and several cytokines/chemokines and their receptors, phosphatases, kinases, and other molecules associated with an inflammatory signature (FIG. 3D; FIG. 6G).

### Example 6: qRT-PCR Analysis of microRNAs

For microRNA profiling, quantitative TaqMan based OpenArray Human MicroRNA Panel (Cat #4470187; Thermo Fisher) was used. This panel contains 754 well-characterized human miRNA sequences from the Sanger miRBase v14. All 754 assays have been functionally validated with miRNA artificial templates. Briefly, total RNA was isolated from normal (n=6) and HS (n=9) skin using mirVana miRNA isolation kit (Cat #AM1561) (FIG. 3E). TaqMan miRNA Reverse Transcription kit (Cat No #4366596, Thermo Fisher Scientific) was used for reverse transcription reaction using megaplex RT and Preamp pool primers v3 (Cat #4444750). Pre-amplification and amplification thermal cycling conditions were used as described in manufacturer's protocol. Expression of miRNAs were read on the 12K Flex RT-PCR machine and relative quantification was done through the −ΔΔCt method. Data analysis was performed through the online available Expression suit (v 1.3) software (Thermo Fisher Scientific) using the global gene normalization method. Bioinformatic analysis of all panels was carried out using IPA (Qiagen, Hilden, DE) (Version 84978992).

A total of 36 differentially expressed miRNAs in HS compared to healthy skin were identified, of which 27 miRNAs were significantly upregulated and 9 miRNAs were significantly down-regulated (log 2FC>|1|, p-value<0.05) (FIG. 3F). Eleven of the 36 differentially expressed miRNAs (miR-150, miR-27a5p, miR-155, miR-21, miR-142-3P, miR-126, miR-29b, Let7, miR-200a) have roles in NK cell differentiation and/or function (FIG. 3G and FIG. 7B). miRNAs that enhance NKT or NK cell differentiation and/or activity (mir-150, miR-155, miR-21, miR-200a, miR29a-5p, Let7) were increased in expression, while those that regulate NK activity (miR-27a-5p, miR-181a-2-3p) were decreased in HS relative to normal controls. Collectively, the cellular landscape, gene expression profiles and miRNA regulome indicate that NK cell populations are critical contributors to the pathogenesis of HS (FIG. 3G).

### Example 7: Sample Processing and Ex Vivo Culture

Clinical specimens were divided into approximately 5 mm×5 mm tissue pieces and placed on to a 0.4 m trans-well filter (Merck Millipore, Burlington, MA) with a thin layer of extracellular matrix (ECM, 90% collagen type 1 (Advanced Biomatrix, Carlsbad, CA)+10% growth factor reduced Matrigel (Corning, Corning, NY). KBM Gold Basal Medium (00192151; Lonza, Basel, CH) containing KGM Gold supplements (00192152, Lonza) was added to the trans-well filters and to the bottom of the well to generate air-liquid (the media volume did not cover the top tissue layer) cultures. Treatment with anti-human CD2 Antibody (10 μg/mL; Cat #300240; Clone: RPA-2.10; Biolegend, San Diego, CA) or vehicle IgG antibody (10 μg/mL, Cat #403502, Clone: QA16A12, Biolegend) were given for 3 days. These treatments were changed daily and at the end of each experiment, a portion of each tissue was fixed for histologic processing or snap frozen in liquid nitrogen for RNA and protein analysis. The supernatant culture media was collected daily and stored at −80° C. for profiling of cytokines, chemokines, and growth factors.

### Example 8: Histological Analysis and Confocal Immunofluorescence (IF) Procedure

Hematoxylin and Eosin (H & E) staining: Briefly, skin tissues were fixed in 10% formalin, embedded in paraffin, and sectioned 5 m thick. The skin sections were deparaffinized in xylene, rehydrated and stained with H & E. Images were captured using a BZ-X710 bright field microscope and integrated for Z-stacking and stitching with BZ-X analyzer (Keyence Corporation, Osaka, JP).

Immunofluorescence ConfocalAnalysis: Skin sections were deparaffinized, rehydrated and then incubated in an antigen unmasking solution according to the manufacturer's instructions (Vector Laboratories, Burlingame, CA). Sections were blocked in blocking buffer containing 5% normal goat serum in PBST (PBS+0.4% tritonX100) for 1 hour at 37° C. Sections were then incubated with primary antibodies against various proteins in blocking solution overnight at 4° C.; antibodies and their dilutions used are listed in Table 3 and Table 4. Sequential staining was done for visualization of more than one target in a single specimen. After washing (3× times, 10 minutes each) with PBST, sections were re-incubated with various fluorescence-coupled secondary antibodies (1:200, Invitrogen). Sections were fixed in DAPI containing Vectashield gold antifade medium (Cat #H-1200, Vector Laboratories, Burlingame, CA) and then visualized under FLUOVIEW FV3000 confocal microscopes (Olympus Center Valley, PA) equipped with FV3000 Galvo scan unit. Z-projected images were post-processed for noise reduction, 3-D image construction, and movie preparation using FV3IS-SW version 2.3.2.169 software provided with the Olympus Fluoview F3000 confocal microscope.

TABLE 3
List of Antibodies Used in Study

| Serial # | Antibody Name | Cat # | Species | Company |
|---|---|---|---|---|
| 1 | Anti-CD3 antibody | ab11089 | Rat | Abcam |
| | [CD3-12] | | monclonal | |
| 2 | Anti-CD3 | ab5690 | Rabbit | Abcam |
| | | | polyclonal | |
| 3 | Anti-CD2 antibody | ab243837 | mouse | Abcam |
| | [OX34] | | monoclonal | |
| 4 | Recombinant Anti-CD2 | ab131276 | Rabbit | Abcam |
| | antibody [EPR6451] | | monoconal | |
| 5 | Anti-NCAM1 antibody | ab118291 | Goat | Abcam |
| | | | polyclonal | |
| 6 | Anti-NCAM1 antibody | ab9272 | Mouse | Abcam |
| | [123C3] | | monoclonal | |
| 7 | Anti-CD58 (LFA3) | 330923 | Mouse | Biolegend |
| | | | monoclonal | |
| 8 | Recombinant Anti-CD4 | ab133616 | Rabbit | Abcam |
| | antibody [EPR6855] | | monoclonal | |
| 9 | CD4 Antibody (GK1.5) | sc-13573 | Rat | Santa Cruz |
| | | | monclonal | Biotechnology |
| 10 | Anti-CD8α | 31-1283-00 | Rabbit | Ray |
| | | | monoclonal | Biosciences |
| 11 | Recombinant Anti-CD68 | ab955 | Mouse | Abcam |
| | antibody [KP1] | | monoclonal | |
| 12 | Recombinant Anti-CD20 | ab78237 | Rabbit | Abcam |
| | antibody [EP459Y] | | monoclonal | |
| 13 | Anti-Human IgG | ab200699 | Mouse | Abcam |
| | antibody [IG266] | | monclonal | |
| 14 | MPO antibody | AF3667 | Goat | rndsystems |
| | | | polyclonal | |
| 15 | F4/80 Monoclonal | 14-4801-85 | Rat | eBiosciences |
| | Antibody (BM8) | | monoclonal | |
| 16 | Anti-Perforin | ab16074 | Rat | Abcam |
| | antibody [CB5.4] | | monoclonal | |
| 17 | Recombinant Anti- | ab209205 | Rabbit | Abcam |
| | Granzyme A | | monoclonal | |
| | antibody [EPR20161] | | | |
| 18 | CD11b Monoclonal | 14-0112-82 | Rat | eBiosciences |
| | Antibody (M1/70) | | monoclonal | |
| 19 | Recombinant Anti- | ab243879 | Rabbit | Abcam |
| | Granzyme B | | monoclonal | |
| | antibody [BLR022E] | | | |
| 20 | Recombinant Anti-PAR2 | ab180953 | Rabbit | Abcam |
| | antibody [EPR13675] | | monoclonal | |
| 21 | Recombinant Anti-CD19 | ab245235 | Rabbit | Abcam |
| | antibody [EPR23174-145] | | monoclonal | |
| 22 | alpha-Smooth Muscle | NB300-978 | Goat | Novus |
| | Actin Antibody | | polyclonal | |
| 23 | Anti-Vimentin | ab8978 | Mouse | Abcam |
| | antibody [RV202] | | monoclonal | |
| 24 | Anti-NKP46 | ab195314 | mouse | R & D |
| | | | monoclonal | system |
| 25 | Anti-CD2 (Clone: | 300239 | Mouse | Biolegend |
| | RPA-2.10) | | IgG1, κ | |
| 26 | IgG1 (CloneQA16A12) | 403502 | Human | Biolegend |
| | | | IgG1, κ | |

TABLE 4
Antibodies and Dilution Used for Immunofluorescence Staining of Each Panel

| | Antibody | Catalogue | | |
|---|---|---|---|---|
| | Name | number | Host | Dilution |
| Staining # 1 | CD2 | ab131276 | Rabbit | 1/250 |
| | | | monoconal | |
| | CD56 | ab9272 | Mouse | 1/500 |
| | | | monoclonal | |
| | CD3 | ab11089 | Rat | 1/100 |
| | | | monclonal | |
| Staining #2 | CD2 | ab131276 | Rabbit | 1/250 |
| | | | monoconal | |
| | CD56 | ab118291 | Goat | 1/100 |
| | | | polyclonal | |
| | CD58 | 330923 | Mouse | 1/50 |
| | | | monoclonal | |
| Staining # 3 | CD2 | ab243837 | mouse | 1/200 |
| | | | monoclonal | |
| | CD56 | ab118291 | Goat | 1/100 |
| | | | polyclonal | |
| | CD3 | ab5690 | Rabbit | 1/100 |
| | | | polyclonal | |
| | Perforin-1 | ab16074 | Rat | 1/100 |
| | | | monoclonal | |
| Staining # 4 | TUNEL | 11684795-910 | Green | Manufacturer's |
| | | | | Proocol |
| | CD2 | ab243837 | mouse | 1/200 |
| | | | monoclonal | |
| | PAR2 | ab180953 | Rabbit | 1/100 |
| | | | monoclonal | |
| Staining # 5 | αSMA | NB300-978 | Goat | 1/100 |
| | | | polyclonal | |
| | CD2 | ab243837 | mouse | 1/200 |
| | | | monoclonal | |
| | CD19 | ab245235 | Rabbit | 1/100 |
| | | | monoclonal | |
| Staining # 6 | Vimentin | ab8978 | Mouse | 1/100 |
| | | | monoclonal | |
| | CD2 | ab131276 | Rabbit | 1/250 |
| | | | monoconal | |
| | CD56 | ab118291 | Goat | 1/100 |
| | | | polyclonal | |
| Staining # 7 | CD2 | ab243837 | mouse | 1/200 |
| | | | monoclonal | |
| | CD56 | ab118291 | Goat | 1/100 |
| | | | polyclonal | |
| | GZMB | ab243879 | Rabbit | 1/250 |
| | | | monoclonal | |
| | CD11b | 14-0112-82 | Rat | 1/100 |
| | | | monoclonal | |
| Staining# 8 | CD2 | ab243837 | mouse | 1/200 |
| | | | monoclonal | |
| | CD56 | ab118291 | Goat | 1/100 |
| | | | polyclonal | |
| | GZMA | ab209205 | Rabbit | 1/100 |
| | | | monoclonal | |
| | CD3 | ab11089 | Rat | 1/100 |
| | | | monclonal | |
| Staining # 9 | CD4 | sc-13573 | Rat | 1/50 |
| | | | monclonal | |
| | CD2 | ab131276 | Rabbit | 1/250 |
| | | | monoconal | |
| | CD68 | ab955 | Mouse | 1/100 |
| | | | monoclonal | |
| Staining # 10 | CD2 | ab243837 | mouse | 1/200 |
| | | | monoclonal | |
| | CD56 | ab118291 | Goat | 1/100 |
| | | | polyclonal | |
| | CD8α | 31-1283-00 | Rabbit | 1/100 |
| | | | monoclonal | |
| Staining # 11 | CD2 | ab243837 | mouse | 1/200 |
| | | | monoclonal | |
| | CD20 | ab78237 | Rabbit | 1/50 |
| | | | monoclonal | |
| | CD3 | ab11089 | Rat | 1/100 |
| | | | monclonal | |
| Staining # 12 | MPO | AF3667 | Goat | 1/100 |
| | | | polyclonal | |
| | F4/80 | 14-4801-85 | Rat | 1/50 |
| | | | monoclonal | |
| | CD4 | ab133616 | Rabbit | 1/500 |
| | | | monoclonal | |

### Example 9: Spatial Localization of NKT and NK Cells in Normal and US Skin

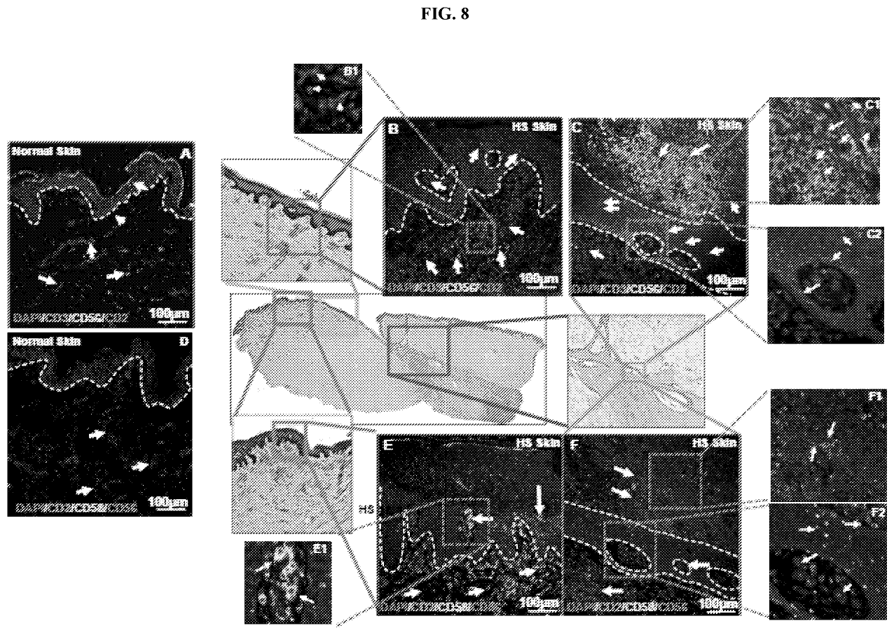

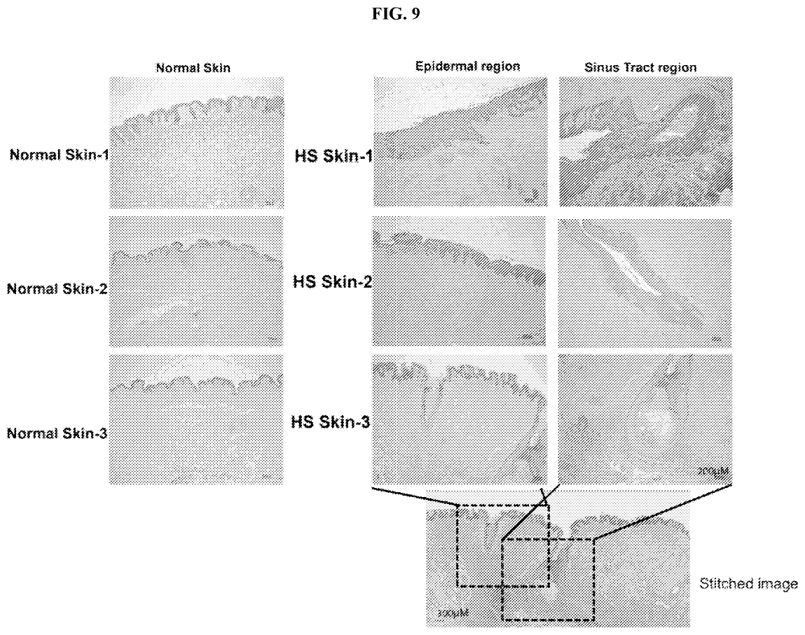

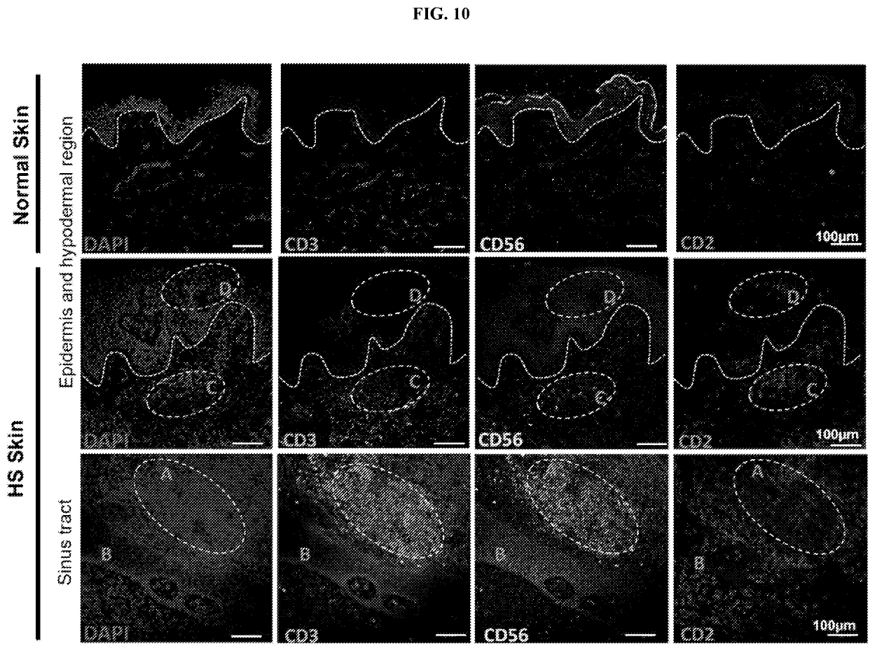

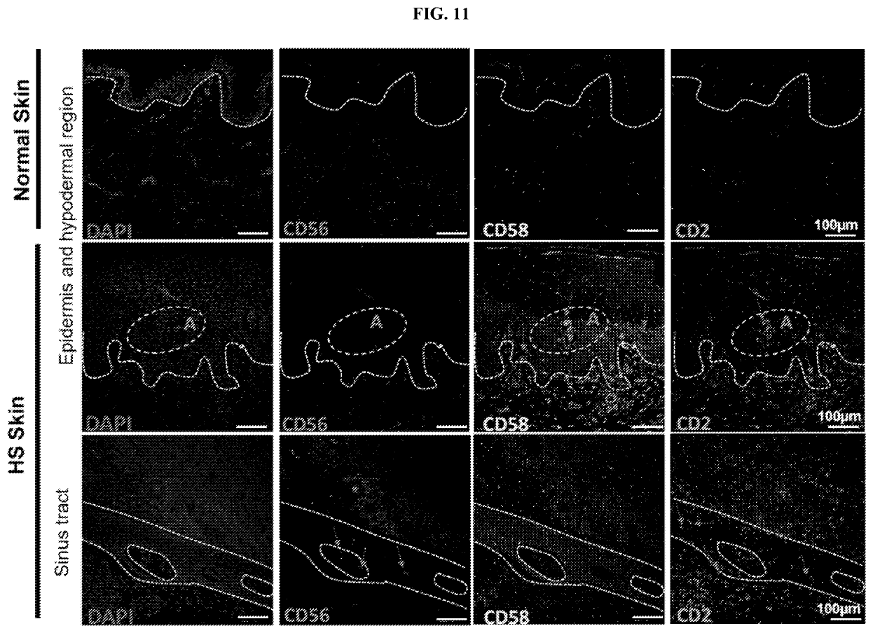

NKT and NIK cells are heterogenous and have functions that are determined by tissue and/or organ localization. Immunofluorescent microscopy was used to correlate their localization to distinct functions in HS pathogenesis. Classical NIK cells include CD56bright (immature) and CD56dim (mature and highly cytolytic), while NKT cells are CD3+CD56bright. H&E stained sections from normal and HS skin were used to localize cells expressing CD56 (pan NIK marker), CD3 (T cells and NKT), CD2 and CD58 (LFA3, ligand of CD2) from immunofluorescent antibody stained serial sections. The tissue morphology of skin from healthy controls revealed well-defined epidermal and dermal regions (FIG. 8A, FIG. 8D, and FIG. 9). In contrast, HS skin (Hurley late stage II and stage III) showed highly inflamed areas with hyperproliferation of the epidermal and dermal compartments (FIG. 8B, FIG. 8C, FIG. 8E, 8F and FIG. 9). Histology showed the presence of sinus tracts protruding deep inside the hypodermis with extensive cellular infiltration of different populations of immune cells (FIG. 9). Control skin contained very few T cells (CD3+CD56−) and NKT or NK cells (mostly CD56bright) (FIG. 8A, FIG. 8B and FIG. 10). In contrast, HS skin was characterized by a high proportion of NK (CD56+), NKT (CD3+CD56+) and T cells (CD3+CD56−) (FIGS. 8C-8F and FIG. 10). In epidermal and dermal regions of HS skin, the NK cells were predominantly CD3-CD56dim, the phenotype of mature cells (FIG. 8C, FIG. 8E and FIG. 10). Whereas in the deep sinus track and near hair follicle regions, the cells were primarily CD56bright NK cells that co-expressed CD3, the characteristics of NKT cells (FIG. 8D, FIG. 8F and FIG. 10). A striking feature was the high expression levels of CD2 on NKT, NK and T cells in HS. The expression of CD58, the ligand of CD2, was significantly elevated on epithelial cells in HS skin but not in control skin. Moreover, these CD58 expressing epithelial cells were juxtaposed to CD2 expressing NK, NKT cells (CD56+) and T cells (CD56−) (FIG. 8B vs FIG. 8E and FIG. 8F and FIG. 11).

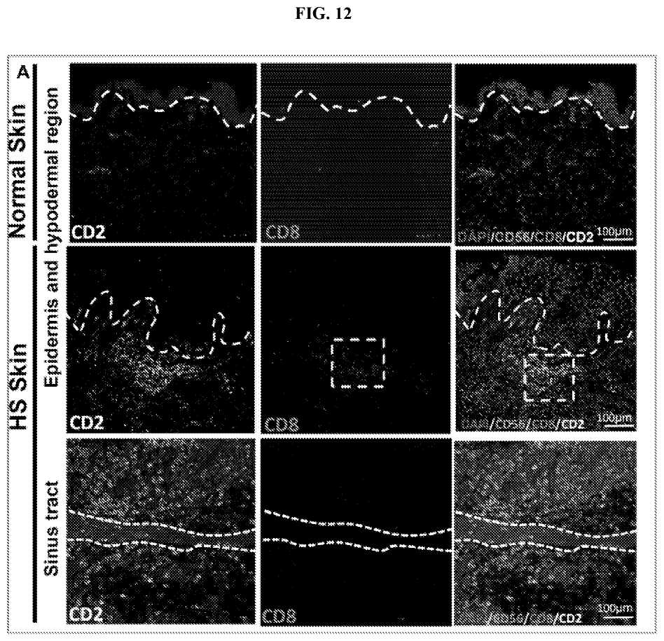

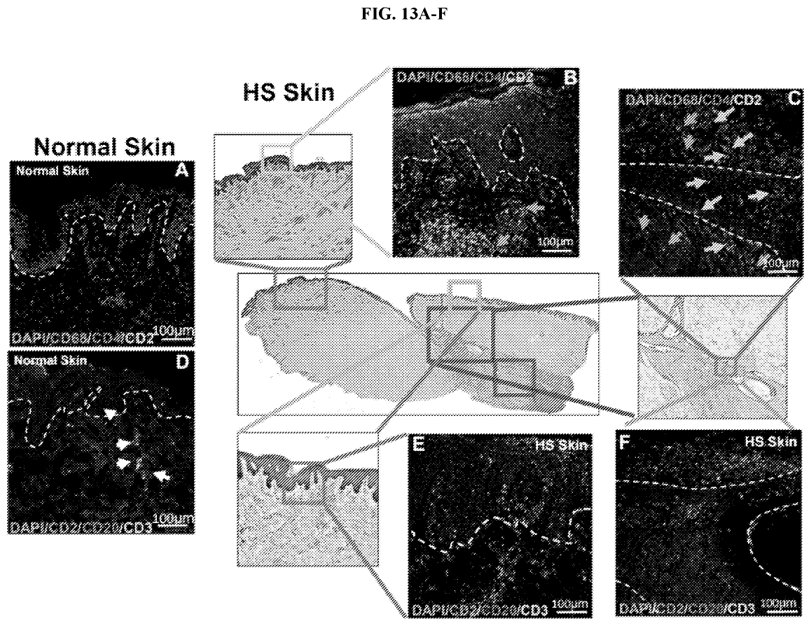

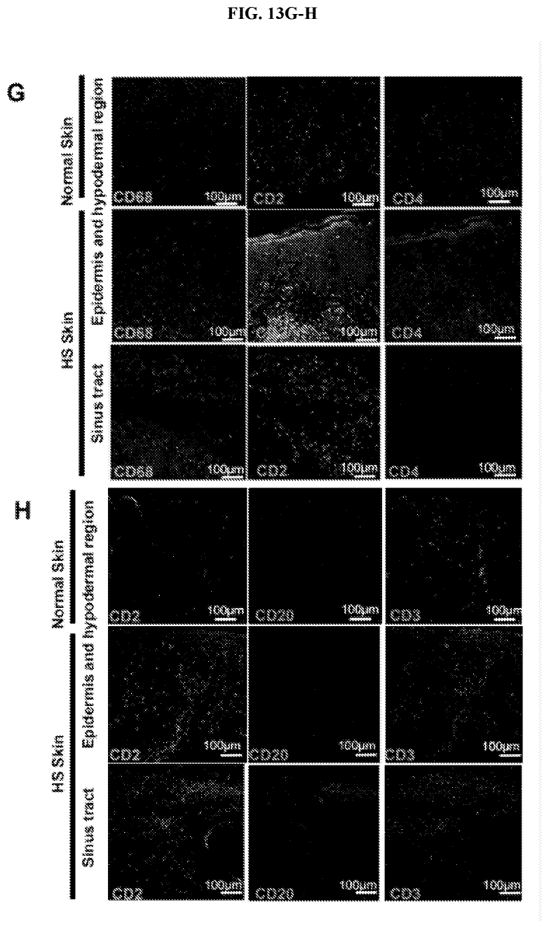

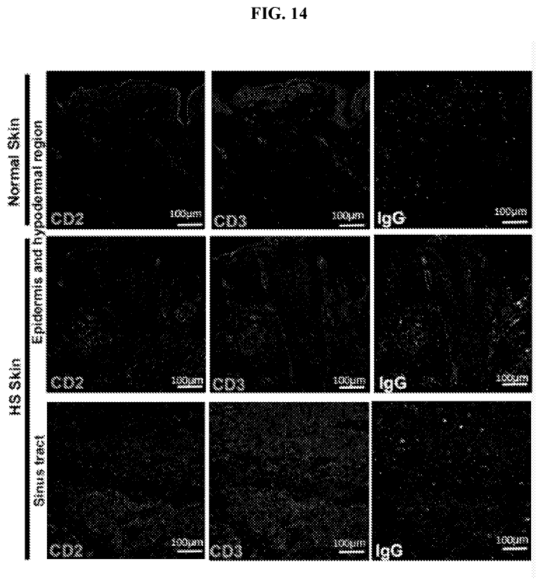

The spatial distribution observed for NKT and NK cell populations was not apparent for T cells and B cell populations (FIG. 12 and FIG. 13). B cells (CD20+) and plasma cells (IgG+) were observed in HS but they did not show a distinct pattern of localization (FIG. 13A-H and FIG. 14). The T cells in epidermal, dermal and sinus tract compartments of HS skin were predominantly CD4+; CD8+ T cells were infrequent (FIG. 12 and FIG. 13). This is consistent with the scRNAseq (FIG. 3A) data. In summary, the data indicated that NKT and NK cells were the major lymphocyte populations within HS skin, and they were spatially localized in distinct regions associated with discrete aspects of disease pathogenesis.

### Example 10: NK and NKT Cell Populations have Distinct Roles in HS Pathogenesis

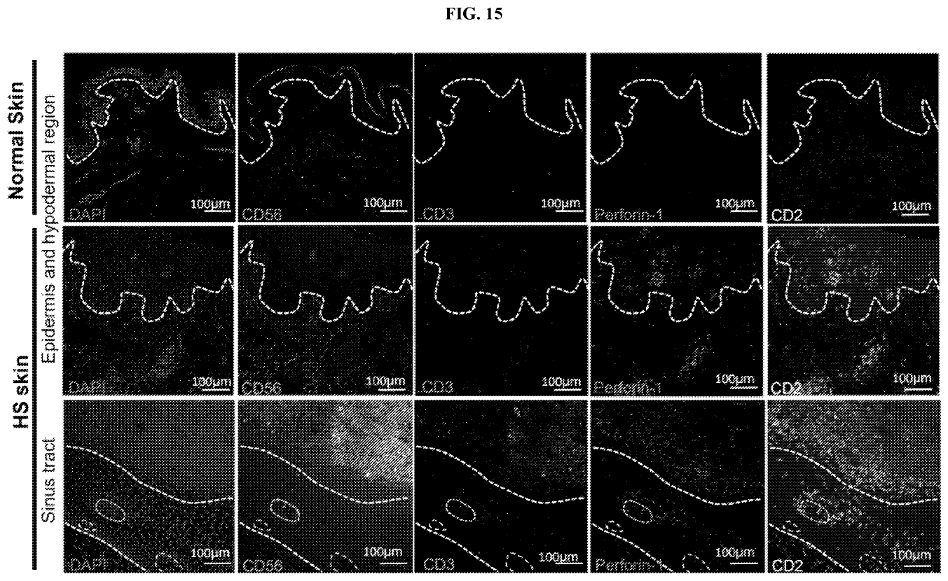

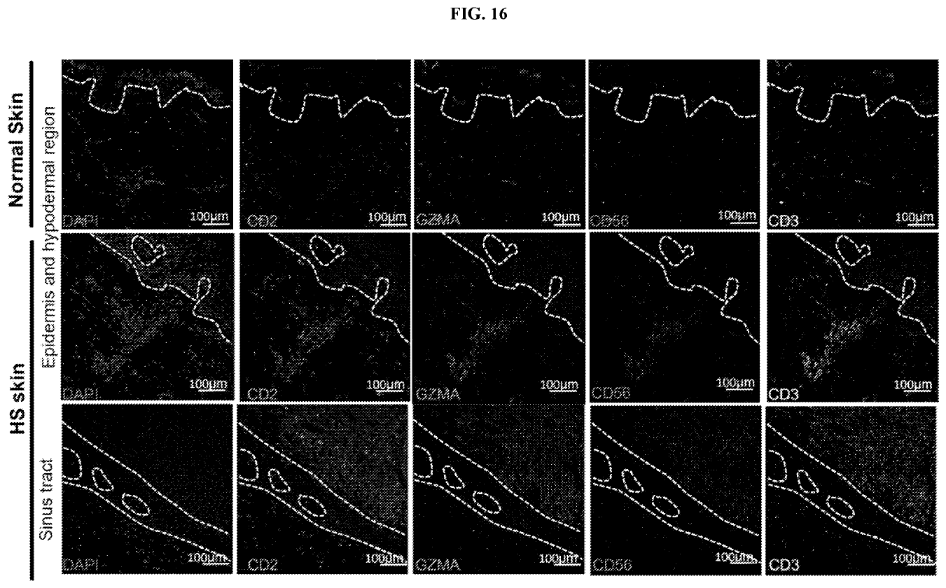

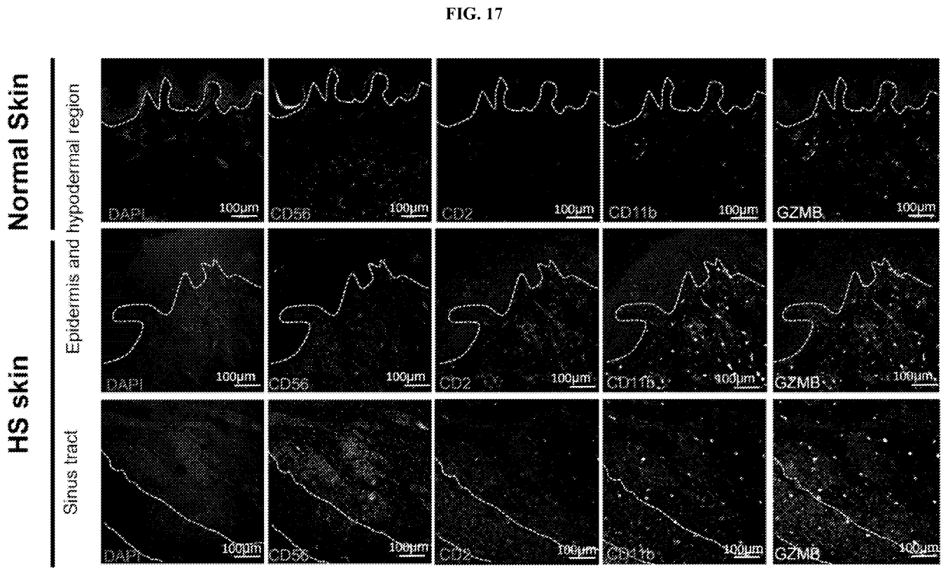

Perforin and granzymes (granzyme A and granzyme B) are proteins necessary for NKT cell and NK cell mediated cytotoxic activity of target cells. Normal skin samples contained a small frequency of perforin-1, granzyme A, and granzyme B expressing cells (FIG. 15, FIG. 16 and FIG. 17). In contrast, HS skin had large numbers of perforin-1, granzyme A and granzyme B expressing classical NK cells (CD3−CD56dim) within epidermal hair follicles, hypodermis and along the border region of sinus tracts that seemed to be linked to the development of sinus tracts (FIG. 15, FIG. 16 and FIG. 17). Whereas NKT and NK cells expressed equivalent levels of granzyme A, the expression of perforin-1 and granzyme B was distinctly lower in NKT cells. The immune-histological staining images show that NK and NKT cells in HS express high levels of CD2, affirming the data from gene expression analysis above. A significant proportion of the CD56dimCD2hi granzyme Bhi cells also expressed high levels of CD11b (MAC1/ITGAM/CR3) (FIG. 17). Overall, the staining patterns reveal a heterogeneity in NKT and NK cell populations in different regions of HS skin.

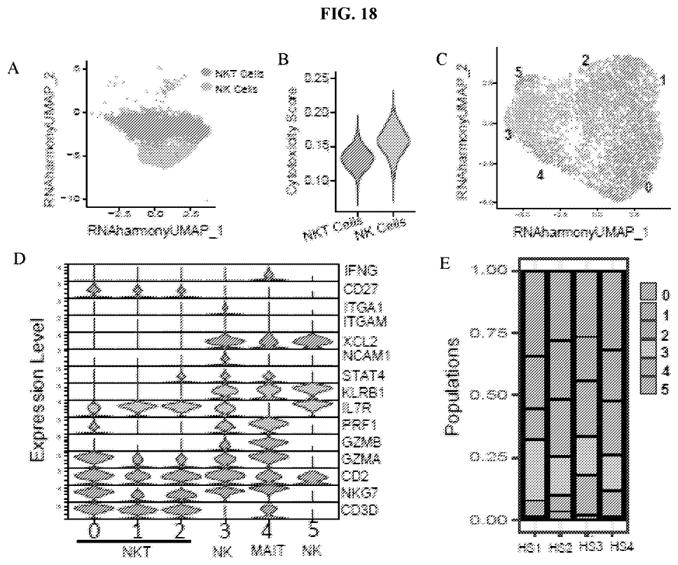

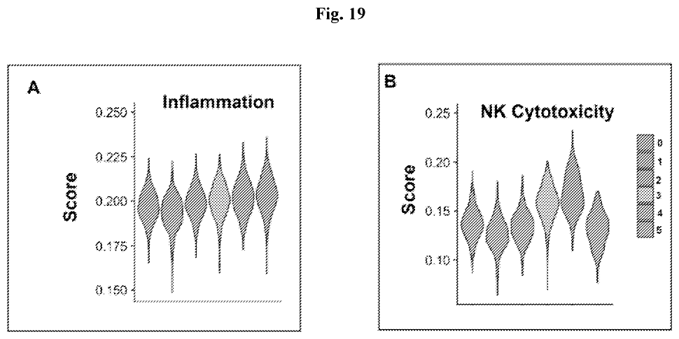

HS scRNAseq data for NKT and NK cells was extracted from FIG. 3A (FIG. 18A). Both NKT and NK cells had significant cytotoxicity scores based on expression of cytotoxicity genes; however, the score of NK cells was higher (FIG. 18B). UMAP clustering revealed 6 populations based on gene expression profiles where three were NKT (CD3+), two were NK (CD3−) and one population had MAIT characteristics (CD3+CD161+) (FIG. 18C and FIG. 18D). These populations of NK cells are present in each of the four HS samples (FIG. 18E). Based on gene expression associated functional scoring, NK populations have similar inflammation scores, whereas cytotoxicity scores were higher in population 3 (NK) and population 4 (MAIT) (FIG. 19).

### Multiplex Analysis

To identify inflammatory cytokine and chemokine responses, ex vivo cultures of HS skin (n=11) or healthy skin tissues (n=13) were exposed to anti-CD2, and multiplex Luminex analysis was performed. Cytokine/chemokine/growth factor 45-Plex Human ProcartaPlex™ Panel 1 (Cat #EPX450-12171-901, Thermo Fisher) was used. Briefly, the tissues were homogenized using cold RIPA buffer (Santa Cruz biotechnologies) containing 2 mM sodium orthovanadate, 1 mM PMSF, and a protein cocktail inhibitor (1×; Santa Cruz). The homogenate is centrifuged at 10,000 rpm for 10 minutes, and the supernatants are stored at −80° C. until assayed. A total of 45 target proteins containing (i) Th1/Th2 markers: GM-CSF, IFNγ, IL-1 β, IL-2, IL-4, IL-5, IL-6, IL-8, IL-12p70, IL-13, IL-18, TNF α (ii) Th9/Th17/Th22/Treg markers: IL-9, IL-10, IL-17A (CTLA-8), IL-21, IL-22, IL-23, IL-27 (iii) Inflammatory cytokines: IFNα, IL-1α, IL-1RA, IL-7, IL-15, IL-31, TNFβ (iv) Chemokines: Eotaxin (CCL11), GROα (CXCL1), IP-10 (CXCL10), MCP-1 (CCL2), MIP-1α (CCL3), MIP-1β (CCL4), RANTES (CCL5), SDF-1 α, and (v) growth factors: BDNF, EGF, FGF-2, HGF, NGF β, PDGF-BB, PlGF-1, SCF, VEGF-A, VEGF-D were assessed. For analysis, 50 μl of conditioned medium was used on the Luminex 200 instrument (Luminex Corporation, USA) as also described earlier (Kashyap M, Kawamorita N, Tyagi V, Sugino Y, Chancellor M, Yoshimura N, et al., J Urol. 2013; 190(2):757-64).

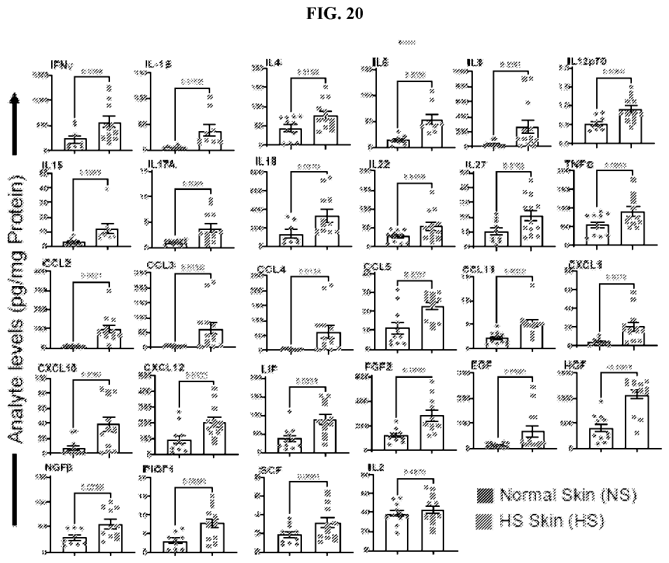

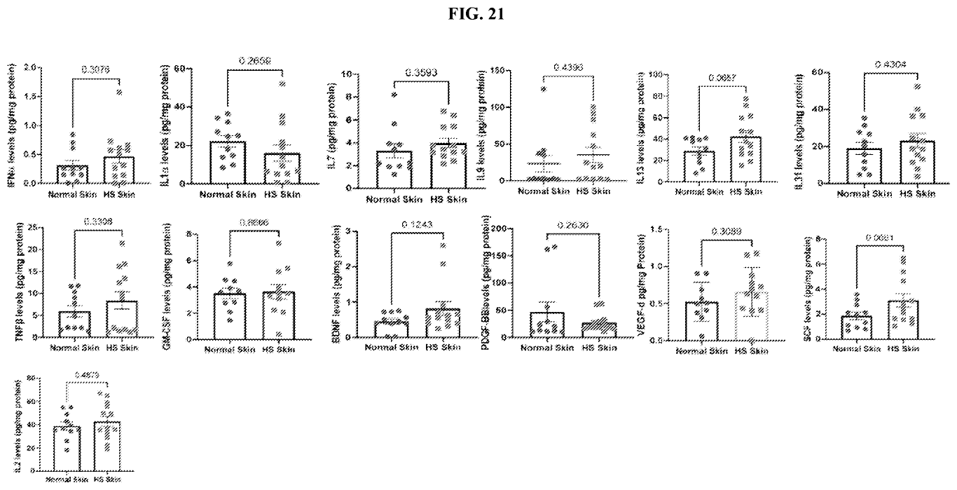

The expression of the cytokines, IFN-γ, IL-1β, IL-4, IL-6, IL-8, IL-12p70, IL-15, IL-17a, IL-18, IL-22, IL-27, and TNF-α were significantly elevated in HS compared to controls (FIG. 20). IFN-γ along with TNF-α were the major effector cytokines expressed by NK and NKT cells. IL-1β induces cytokine/chemokine production by keratinocytes, IL-8 (CXCL8), which plays an important function in recruiting neutrophils, and both are significantly enhanced in HS. Keratinocytes are the primary expressors of IL-15 and IL-18 which were highly elevated in HS skin. These cytokines enhance NK cell cytotoxic activity as well as induce the expression of CD2 and thus play a role in NKT cells and classical NK cell mediated HS pathogenesis. IL-12 is elevated in HS and importantly, promotes survival and expansion of NK cells independent of IL-15 and IL-18. The levels of IFN-α, IL-1α, IL-2, IL-7, IL-9, IL-31, and TNF-β were similar in HS and control skin (FIG. 21). IL-13 was elevated in HS skin, but its levels were not statistically different from that of the controls (FIG. 21). Several chemokines and growth factors were elevated in HS skin vs controls. These included CCL2 (MCP1), CCL3 (MIP-1α), CCL4 (MIP-1β), CCL5 (Rantes), CCL11 (Eotaxin), CXCL1 (GROα/KC), CXCL9, and CXCL10 (IP10), CXCL12 (SDF-1), NGFβ, EGF, FGF-2, HGF, and PIGF1 (FIG. 20). The growth factors BDNF, PDGF-BB, PIGF-1, VEGF and SCF, were present in HS skin but they were not significantly changed from controls (FIG. 21). An intricate interplay of cytokine, chemokines, and growth factors produced by NK cell populations, keratinocytes, and HS cell populations perpetuate the pathogenic machinery of the disease. The results show that the upregulation of CD2 on all NK cell populations and T cells is a key feature of HS. These data demonstrate that a CD2 blockade in HS can reverse the expression of disease associated cytokines, chemokines, and growth factors as well as the gene expression signature.

### Testing CD2 Blockade in Organotypic Cultures

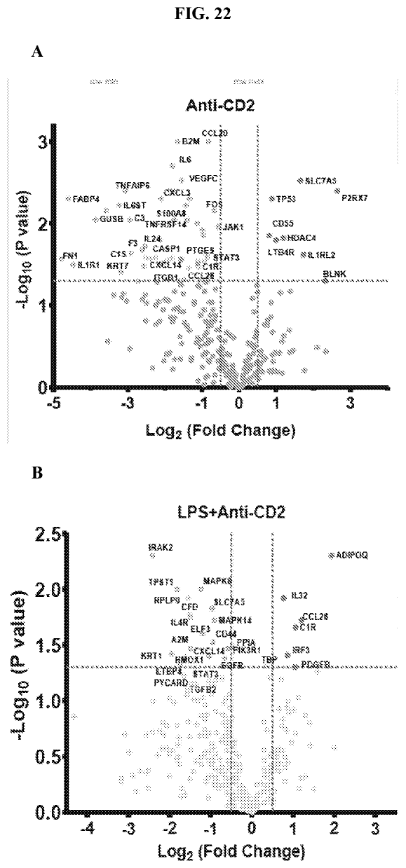

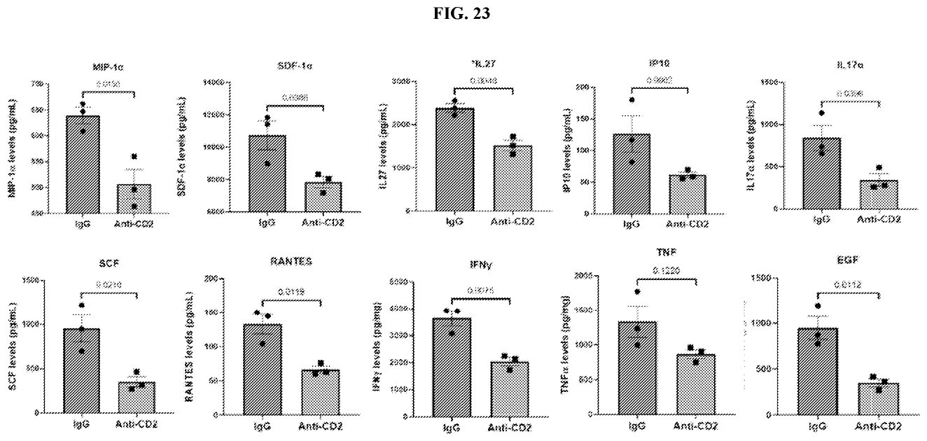

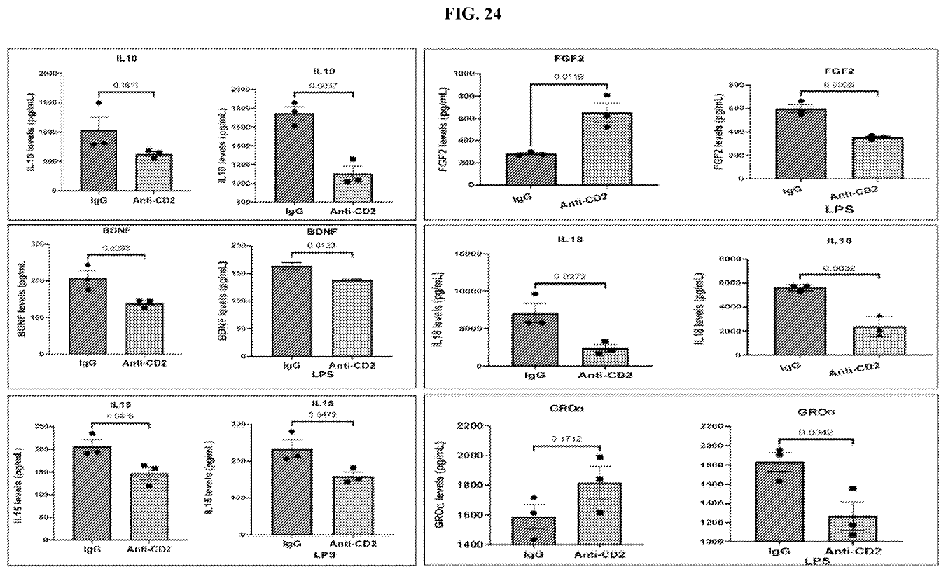

HS skin organotypic cultures (Goliwas K F, Kashyap M P, Khan J, Sinha R, Weng Z, Oak A S W, et al., Inflammation. 2022; 45(3):1388-401) were treated with either anti-CD2 mAb vs. IgG (control) or anti-CD2 mAb vs IgG along with LPS. Cultures treated with anti-CD2 mAB vs. IgG alone displayed changes in gene expression, as measured by inflammatory gene expression arrays. Genes associated with NK and T cell activation and effector function were down regulated following treatment with anti-CD2 (FIG. 22A and FIG. 23). Anti-CD2 also reversed the pathogenic cytokine expression pattern and gene expression signature in LPS-treated HS skin cultures. LPS is a bacterial component which is known to induce various innate immune responses. Specifically, anti-CD2 treatment led to decreased expression of LPS-induced cytokines/chemokines/growth factors and gene signature associated with TLR2/4 signaling. These include IL-10, IL-15, IL-18, FGF-2, IRAK2, MAPK, ILFR and others (FIG. 22B and FIG. 24).

### Pathway Enrichment Analysis

The canonical pathway analysis on DEGs was performed using Ingenuity Pathway Analysis (IPA) (Kramer A, Green J, Pollard J, Jr., and Tugendreich S., Bioinformatics. 2014; 30(4):523-30) with significant fold change and test statistics values. Additional function enrichment analysis was performed by enrichR (Chen E Y, Tan C M, Kou Y, Duan Q, Wang Z, Meirelles G V, et al., BMC Bioinformatics. 2013; 14:128), and metascape (Zhou Y, Zhou B, Pache L, Chang M, Khodabakhshi A H, Tanaseichuk O, et al., Nat Commun. 2019; 10(1):1523) pathway analysis platforms with standard significance (q-value<0.05). The disease enrichment analysis was performed through enrichR with standard significance (q-value<0.05).

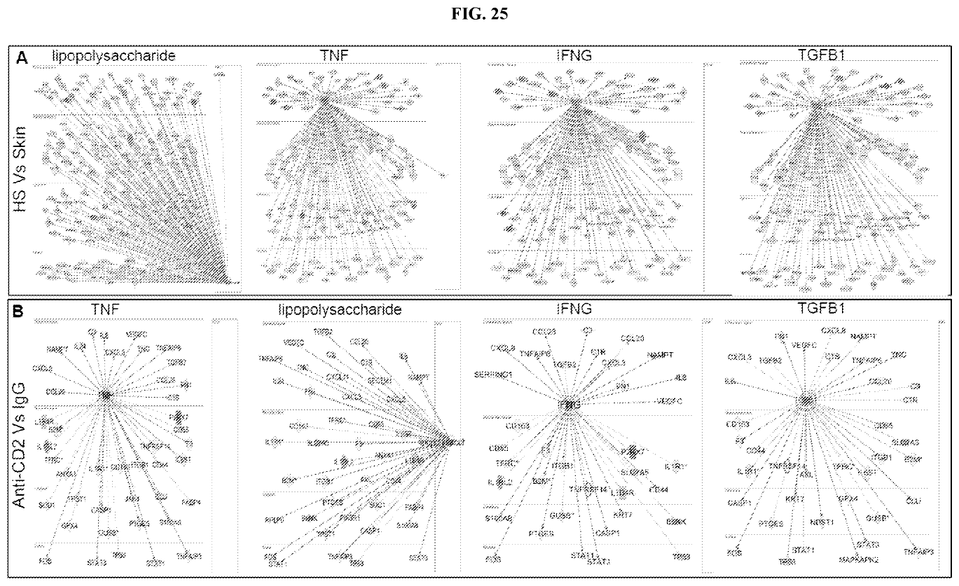

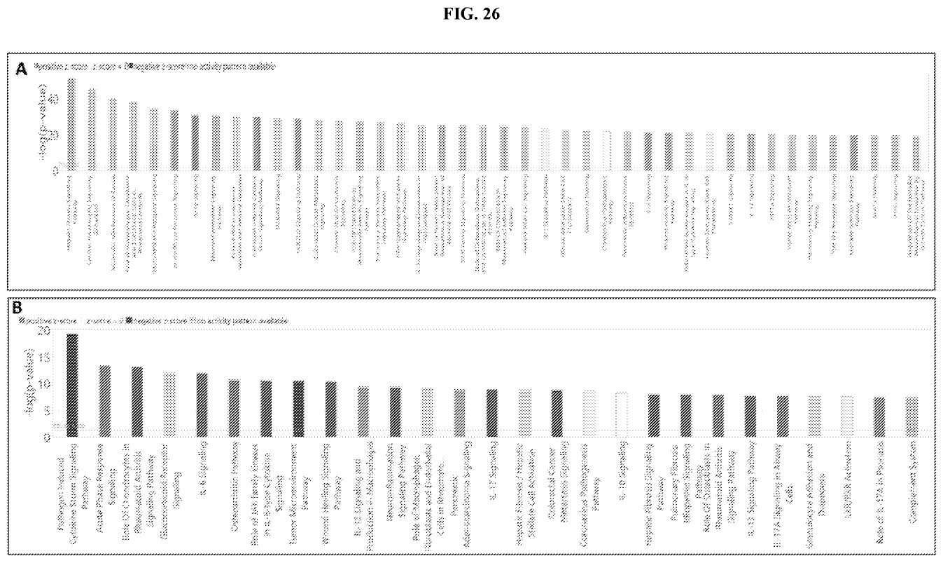

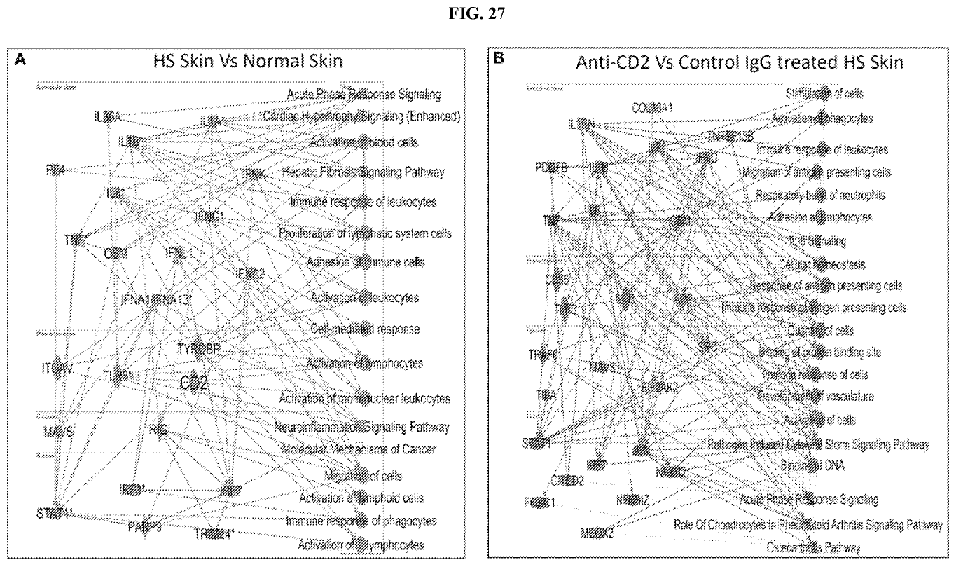

To determine which signaling pathways are down regulated in HS by the CD2 blockade, IPA analyses was applied using qRT-PCR HS vs normal skin array data (FIG. 3D) and anti-CD2 vs IgG array data. The four top ranking upstream regulatory drivers in HS were Lipopolysaccharide (P≤8.27E-81, z-score=6.550), TNF (P≤6.13E-70, z-score=4.994), IFNG (P≤3.96E-64, z-score=5.459), and TGFB1 (P≤1.12E-60, z-score=3.963) (FIG. 25A). These were also the top four ranking regulatory drivers that were down regulated by anti-CD2 treatment, TNF (P≤1.70E-32, z-score=−3.721), lipopolysaccharide (P≤1.61E-28; z-score=−4.288), IFNG (P≤1.72E-27; z-score=−2.993) and TGFB1 (P≤3.52E-26, zscore=−2.236) (FIG. 25B). The majority of the signaling pathways upregulated in HS vs normal skin (FIG. 26A and FIG. 27A) were down regulated by the CD2 blockade in organotypic cultures (FIG. 26B and FIG. 27B).

### Visualizations

Single cell RNA seq analysis plots (clusters, heatmaps, dot plot, cell-cell communication) were generated through R (v4.0.3) statistical software. All experimental verifications' violine plots, volcano plots, and network centrality comparison violine plots were drawn using GraphPad Prism.

### Statistical Analysis

To identify the marker genes in single cell RNA-seq clusters, a nonparametric Wilcox test was used with default parameters. To identify the DEGs in bulk transcriptome data sets, a standard cutoff (FDR≤0.05; log 2fc≥|1|) was used. The activated and inhibited canonical pathway analysis was determined by parameters (z-score≥|1|; BH p-value≤0.05). The significance of experimentally verified DEGs was (students T. test (p-value)≤0.05 and log 2fc≥|1|). The functional enrichment of genes/proteins was performed with significance (−log 10(q-value>1.3). The network power law distribution cutoff was significance (R2>0.7; students t-test p-value≤0.05). The pairwise network centrality distribution comparison significance was achieved through (welch t-test≤0.05). The correlation between two network centralities were calculated by R (v4.0.3). The top 5% nodes with high centralities were selected as a significant protein candidate. The cutoff of proteins to be between inner layer was among the 90^{th} percentile of shells. The centrality enrichment significance was tested by hypergeometric test p-value≤0.05. To identify the DEMs in miRNAome datasets a standard cutoff (p-value≤0.05; log 2fc≥|1|) was used.

1. A method for treating or preventing an inflammatory skin disorder in a subject comprising administering to a subject having or at risk of developing an inflammatory skin disorder an agent that inhibits NK cell activity.

2. The method of claim 1, wherein the inflammatory skin disorder is hidradentitis suppurativa (HS).

3. The method of claim 1, wherein the agent that inhibits NK cell activity blocks the interaction of CD2 and CD58.

4. The method of claim 3, wherein the agent that blocks the interaction of CD2 and CD58 is an anti-CD2 agent.

5. The method of claim 4, wherein the anti-CD2 agent is an anti-CD2 antibody.

6. The method of claim 5, wherein the anti-CD2 antibody is Siplizumab.

7. The method of claim 1, wherein the agent that blocks the interaction of CD2 and CD58 is an anti-CD58 agent.

8. The method of claim 7, wherein the anti-CD58 agent is an anti-CD58 antibody.

9. The method of claim 1, further comprising administering an anti-IL-15 agent, an anti-IL-18 agent, and/or an anti-fibrotic agent.

10. The method of claim 1, wherein the subject's inflammatory skin disorder is refractory to one or more previous treatments of the inflammatory skin disorder, wherein the previous treatment was not an agent that blocks the interaction of CD2 and CD58.
